(19) United States
(12) Patent Application Publication
(10) Pub. No.: US 2026/0306403 A1
(43) Pub. Date: Oct. 8, 2026

(54) MODIFIED LIPOSOME FOR DRUG DELIVERY, USE OF LIPOSOME FOR DRUG DELIVERY, ESPECIALLY WITH ANTI-CANCER ACTIVITY, AND WAYS TO LOAD LIPOSOME WITH DRUGS, ESPECIALLY WITH ANTI-CANCER ACTIVITY

(71) Applicant: BS BIOTECHNA SPOLKA AKCYJNA, Krakow (PL)

(72) Inventors: Olga SWIECH, Bieniewice (PL); Agata KRZAK, Warszawa (PL); Joanna PIETRAS, Milanowek (PL); Weronika PIOTROWSKA, Warszawa (PL); Anna BOGUSZEWSKA-CZUBARA, Lublin (PL); Mateusz PSURSKI, Wroclaw (PL); Joanna WIETRZYK, Wroclaw (PL); Ewa OLISZEWSKA, Wroclaw (PL); Joanna JAROSZ, Wroclaw (PL); Wojciech SMORON, Krakow (PL); Renata BILEWICZ, Warszawa (PL)

(21) Appl. No.: 19/480,988

(22) PCT Filed: May 3, 2024
(86) PCT No.: PCT/PL2024/000024

§ 371 Date: Nov. 3, 2025

(30) Foreign Application Priority Data
May 3, 2023 (PL) P.444722

(51) Int. Cl. A61K 9/1271; A61K 9/1278; A61K 31/136; A61K 31/4745; A61K 31/553

(52) U.S. Cl. CPC A61K 9/1271; A61K 9/1278; A61K 31/136; A61K 31/4745; A61K 31/553

(57) ABSTRACT

A modified liposome for drug delivery characterised in that it is composed of the three main lipids DPPC, DSPE-PEG(2000)amine and DOPC together with its modifications and its use for the delivery of at least one drug, in particular with anticancer activity, and a method of loading the liposome with drugs, in particular with anticancer activity.

The object of the invention is a modified liposome for drug delivery, the use of a liposome for drug delivery, in particular with anti-cancer effects, and methods of loading the liposome with drugs, in particular with anti-cancer effects.

Liposomes are commonly used as carriers in chemotherapy with cytostatic drugs, due to their ability to transport these drugs to their target sites in the body. They are spherical vesicles made up of one or more phospholipid bilayers surrounding a liquid interior. The lipid composition of liposomes is crucial for their stability and physicochemical properties. The choice of suitable lipids and their ratio affects the size, shape, stability and surface properties of liposomes. The size and surface properties of liposomes, on the other hand, are crucial for their bioavailability, stability and their selectivity of drug delivery to target tissues and cells (https://doi.org/10.1016/j.addr.2012.09.037). Polyethylene glycol (PEG) is commonly used in the surface modification of liposomes to increase their stability in plasma, improve bioavailability and prolong circulation time (DOI: 10.1016/j.jconrel.2013.07.026). In addition, the chain length of polyethylene glycol (PEG) can affect the site of drug accumulation in the human body and the targeting efficiency of a therapy (10.1016/j.biomaterials.2011.04.082). The most commonly used polyethylene glycol for modifying liposomes is a polymer terminated with a terminal methoxyl group (—O—CH₃), the so-called MPEG. Other modifications, such as a terminal amino group (—NH2) or a carboxyl group (—COOH), are usually used as intermediates for further modification with proteins or other cell homing molecules (https://doi.org/10.1038/s41598-021-86860-5) and their potential to generate surface charge in the changing tumour environment has so far been neglected. On the other hand, it is known that the surface charge of liposomes can influence where they accumulate in the body and, in particular, their accumulation in tumour tissues. Studies show that positively charged liposomes have better accumulation and penetration of the carrier in tumour tissues and better cellular uptake compared to inert and negatively charged nanoparticles (https://doi.org/10.1016/j.nantod.2016.04.008). The presence of a positive charge on the surface of liposomes is therefore crucial in ensuring efficient and specific transport into tumour tissues.

Emetine is a low-molecular-weight drug showing anticancer activity. The drug has been tested in phase I and II clinical trials as a separate chemotherapeutic agent and also in combination with other therapeutics. Studies have shown that the therapeutic index of emetine is very limited and that increasing the dose resulted in cardiac toxicity, which has inhibited further development of work on the use of emetine in anticancer therapy. Despite this, a number of studies indicate the high potential of emetine in the treatment of a number of cancers with a particular focus on breast cancer (doi: 10.17179/excli2016-280; DOI: 10.3892/or.2019.7290). There is therefore a great need to search for drug carriers capable of efficiently binding and transferring emetine. Previous studies have shown that emetine, despite its slight alkaline properties, loads into lipid carriers using a pH and concentration gradient of only 50%. In addition, during loading, the Zeta potential of liposomes increases significantly suggesting that emetine binds to the liposome surface rather than loading the drug into the liposome (DOI:10.1016/j.ejpb.2014.04.002). The surface interaction, however, results in rapid drug shedding in the presence of plasma proteins, and thus a lack of protective effect of the carrier and high toxicity of the therapy.

The essence of the solution according to the first invention is that the liposome is composed of three main lipids, namely DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine in an amount of 10.6 to 10.7% by weight, and at least one lipid from the group comprising DOPC(dioleoylphosphatidylglycerol) and DOPG(dioleoylphosphatidylcholine), the DOPC(dioleoylphosphatidylglycerol) content of the liposome being not more than 22.4% by weight and the DOPG(dioleoylphosphatidylcholine) content of the liposome being not more than 22.6% by weight.

Advantageously, the DOPC (dioleoylphosphatidylglycerol) content of the liposome ranges from 7.5 to 22.4 wt %.

Advantageously, the content of DOPG (dioleoylphosphatidylcholine) in the liposome ranges from 7.5 to 22.6 wt %.

Advantageously, the outer surface of the liposome is coated with a layer of human serum albumin (HSA).

The essence of the solution according to the invention in terms of application is that a liposome composed of at least three lipids, including DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine in an amount of 10.6 to 10.7% by weight is used to carry at least one drug, in particular one with anticancer activity.

Advantageously, the liposome contains DOPC (dioleoylphosphatidylglycerol) in an amount of not more than 22.4 percent by weight.

Advantageously, the liposome contains DOPG (dioleoylphosphatidylcholine) in an amount of not more than 22.6 percent by weight.

Advantageously, the outer surface of the liposome is coated with a layer of human serum albumin (HSA).

Advantageously, the liposome is used to carry emetine.

Advantageously, the liposome is used to carry mitoxantrone.

Advantageously, the liposome is used to carry staurosporine.

Advantageously, the liposome is obtained by preparing a solution of a lipid mixture containing DPPC (1,2-dipalmitoyl-sn-glycero-3-phosphocholine) at a concentration of 17.0 mg/ml to 70.7 mg/ml, cholesterol at concentrations from 6.4 mg/ml to 18.6 mg/ml, DSPE-PEG(2000)amine (ammonium salt of 1,2-distearoyl-sn-glycero-3-phosphoethanolamine-N-[amino(polyethylene glycol)-2000]) from 3.7 mg/ml to 10.7 mg/ml and DOPC (dioleoylphosphatidylglycerol) from 0 mg/ml to 15.7 mg/ml and DOPG (dioleoylphosphatidylcholine) from 0 mg to 15.8 mg/ml in ethanol, heating the solution at 60° C. for 15 minutes, preparing an ammonium sulphate solution of 250 mM to 350 mM in water, fixing the pH between 2.62 and 5.5 by titrating with 0.2 M NaOH solution and finally mixing the lipid solution in ethanol with the ammonium sulphate solution in a volume ratio of 1:5 to 1:9.

Advantageously, hydroxypropyl cyclodextrin (HPCD) at a concentration of 10 mg/ml is added to the ammonium sulphate solution.

Advantageously, the outer surface of the liposome is coated with a layer of human serum albumin (HSA).

The essence of the solution of the first method is that to liposomes composed of at least three lipids, including DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine (ammonium salt of 1,2-distearoyl-sn-glycero-3-phosphoethanolamine-N-[amino(polyethylene glycol)-2000]) in an amount of 10.6 to 10.7% by weight, at least one drug is introduced in such a way that the liposomes are dialysed in buffer 0.1 M PBS pH 7.4 to 9.0 for 12 h to 24 h to obtain a pH and concentration gradient, a solution of at least one drug in DMSO or water of 50 mg/ml is added sequentially for every 1 ml of liposome solution. The solution is cooled to 4° C. and allowed to stand for 4 h to 14 h or heated in a water bath for 15 to 60 minutes at 45 to 60° C. The resulting nanoparticles are then dialysed in PBS buffer pH 7.4 at 4° C. or purified on a chromatography column. Subsequently, the drug concentration and the encapsulation rate of the drug into the liposomes are determined.

Advantageously, the liposome contains DOPC (dioleoylphosphatidylglycerol) in an amount of no more than 1.77 mg/ml.

Advantageously, the liposome contains DOPG (dioleoylphosphatidylcholine) in an amount of no more than 1.58 mg/ml.

Advantageously, the drug is emetine, introduced into solution as emetine hydrochloride at a concentration of 50 mg/ml in an amount of 5 to 80 ul per 1 ml of liposome solution.

Advantageously, the drug is mitoxantrone, introduced into solution as mitoxantrone dihydrochloride at a concentration of 50 mg/ml in an amount of 12.5 ul to 30 ul per ml of liposome solution.

Advantageously, after loading the drug into the liposomes, a 10% aqueous solution of HSA is added at a volume ratio of liposomes to HSA solution of 3:1.

Advantageously, the dialysis of the nanoparticles is carried out on a Sephadex G-25 or G-50 packed chromatography column.

Advantageously, the drug concentration and the encapsulation rate of the drug into the liposomes are determined by UV-Vis spectroscopy, HPLC with UV-Vis detection or by LC-MS.

Advantageously, the drugs are stabilised with hydroxypropyl cyclodextrin (HPCD).

The essence of the solution according to the second invention of the method is that to liposomes composed of at least three lipids including DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine (ammonium salt of 1,2-distearoyl-sn-glycero-3-phosphoethanolamine-N-[amino(polyethylene glycol)-2000]) in an amount of 10.6 to 10.7% by weight, at least one drug is introduced, the primary drug being staurosporine, such that the liposomes are dialysed in 0.1 M acetate buffer, pH 4.5 to 5.5, from 2 h to 12 h at 4° C. to replace the external buffer, and then 20 to 50 ul of DMSO for each 1 ml of liposomes and 3.76 ul to 40 ul of a staurosporine solution of 40 mg/ml in DMSO are added to the liposomes in divided doses, stirring each time until the precipitate disappears. The solution is then left at room temperature for 15 to 60 minutes. Excess staurosporine is purified by dialysis in 0.1M PBS buffer, pH 7.4 to 8.5, changing the external buffer three times after 1 h, 6 h, 12 h.

Advantageously, the liposome contains DOPC (dioleoylphosphatidylglycerol) in an amount of no more than 1.77 mg/ml.

Advantageously, the liposome contains DOPG (dioleoylphosphatidylcholine) in an amount of no more than 1.58 mg/ml.

Advantageously, after loading staurosporine into the liposomes, a 10% aqueous solution of human serum albumin (HSA) is added at a volume ratio of liposomes to HSA solution of 3:1.

Advantageously, the concentration of loaded staurosporine is determined by UV-Vis spectroscopy against a calibration curve by breaking up a sample of liposomes by the addition of 0.3M HCl in a volume ratio of 1:1 and successively 18 times the volume of ethanol.

Advantageously, an emetine hydrochloride solution of 50 mg/ml in water or DMSO is added to the liposomes containing staurosporine after dialysis in PBS buffer in an amount of 10 ul to 33.4 ul per 1 ml of liposomes and then left for 6 h to 12 h at 4° C. or heated for 15 minutes at 45° C.-60° C., after which the % EE encapsulation rate of both drugs is determined taking advantage of the poor water solubility of staurosporine by separating the liposomes on a chromatography column by eluting the unbound drugs from the liposome successively with PBS buffer pH 7.4 and a solution of water:ethanol (1:1). The remaining liposomes are purified by dialysis in 0.1 M PBS buffer or saline and the concentration is determined by UV-Vis spectroscopy against a calibration curve, or HPLC with UV detection, or by LC-MS.

Advantageously, staurosporine is stabilised with hydroxypropyl cyclodextrin (HPCD).

The main advantage of modified liposomes as drug carriers is that, due to the positive charge generated by the presence of the terminal amino group, there is an effective accumulation of the carrier with the drug in solid tumours, and thus anticancer therapy with these drugs is much more effective. This was demonstrated in in vitro studies for nanoparticles loaded with emetine and mitoxantrone and mitoxantrone alone. In the case of these studies, a 3-4 times higher treatment efficacy was obtained, resulting in tumour shrinkage at twice the dose compared to the free drugs.

The developed carriers allow emetine loading of up to above 95% for a drug to lipid ratio of 0.1, which is twice the value reported in the literature. At higher initial drug concentrations, liposomes allow up to four times higher drug to lipid ratios than liposomes reported in the publications. The reason for such good loading is the specific lipid structure containing a terminal amino group in the polyethylene glycol molecule.

These carriers ensure that emetine is loaded inside the liposomes and not deposited on the surface, as indicated by the lack of significant changes in the Zeta potential values of the liposomes after the drug loading process. Additional studies of emetine-loaded liposomes in the presence of human and rat albumin confirm the lack of sudden drug ejection under in vitro test conditions.

These carriers also allow the binding of emetine in the presence of other drugs such as mitoxantrone and staurosporine without a drastic decrease in the % EE encapsulation value of emetine. The presence of a 5-fold excess of mitoxantrone (a drug from the anthracycline group) preserves the % EE of emetine at 95-99%, whereas literature values of liposomal carriers containing an anthracycline-emetine mixture at these concentrations indicate a decrease in % EE to values lower than 30%.

The carriers also provide a significant reduction in the toxicity of emetine encapsulated in liposomes compared to free emetine. They indicate much lower LC50 values for drugs encapsulated in carriers. The LC50 value calculated for emetine encapsulated in a liposome is 12 times higher after 24 h of dosing than the value calculated for the free drug. This creates the possibility of significantly reducing the toxicity of this drug and thus enabling the use of this drug in anticancer therapies.

The solutions according to the inventions are illustrated by the following manufacturing examples and drawing, where:

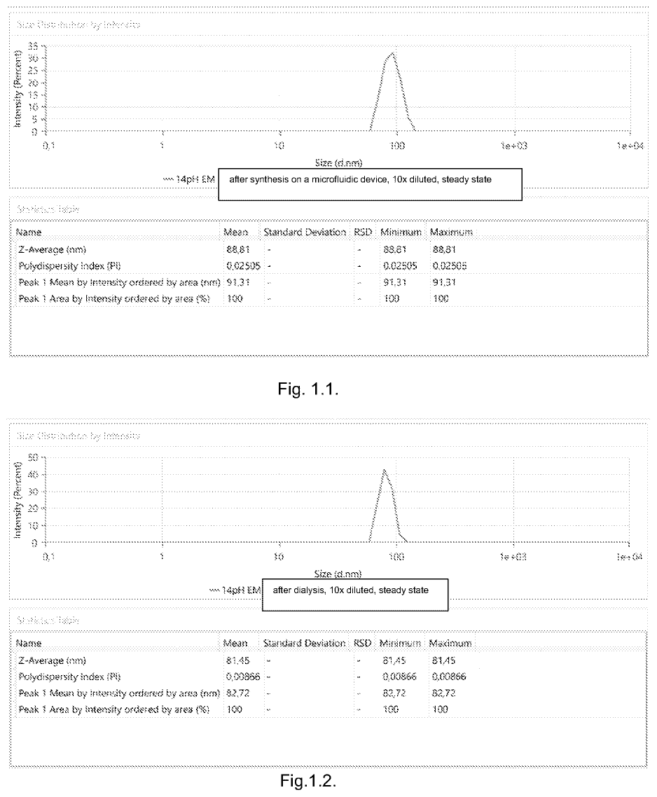

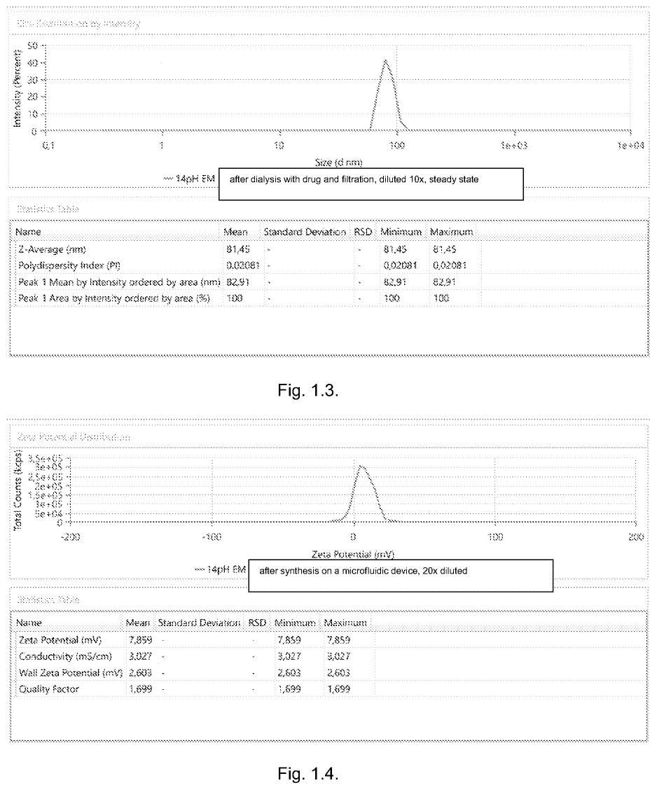

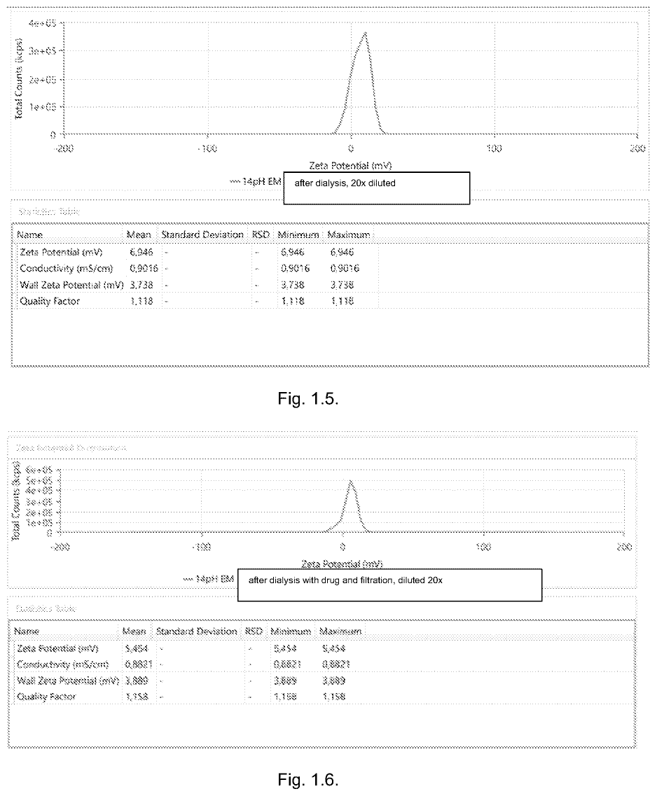

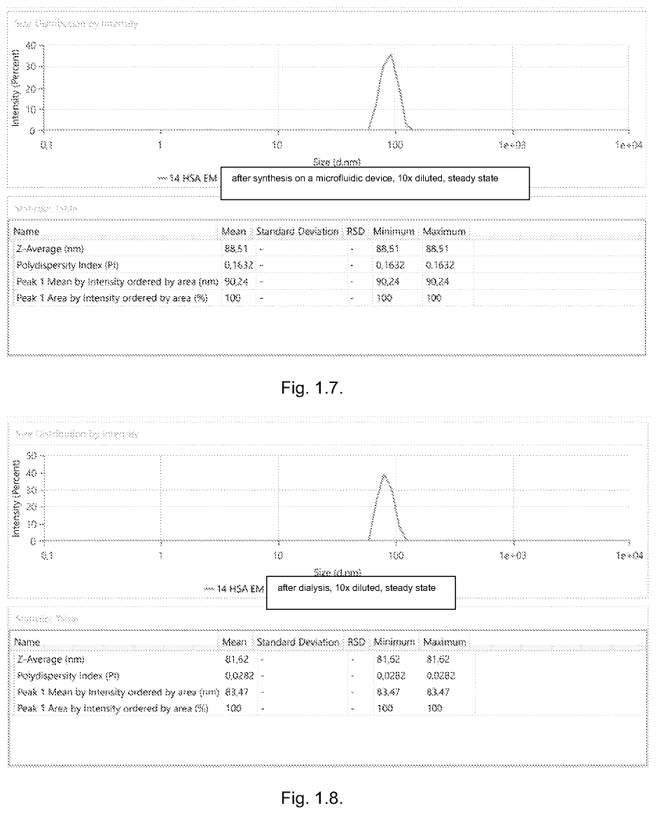

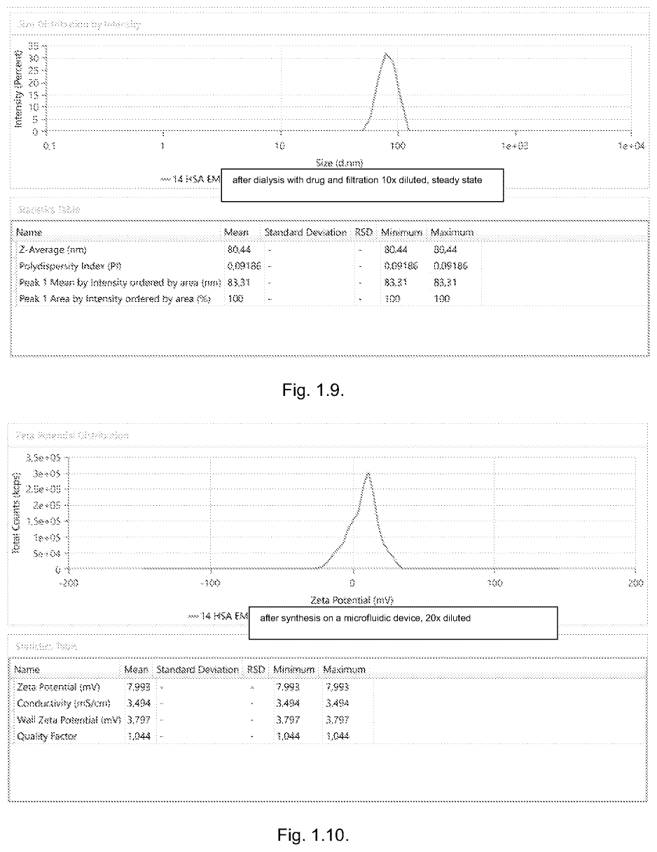

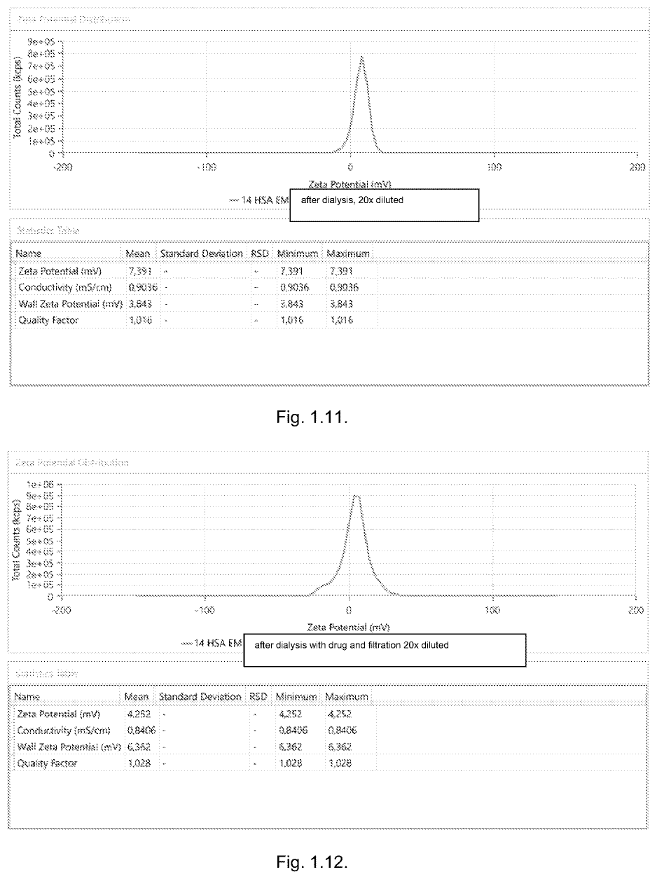

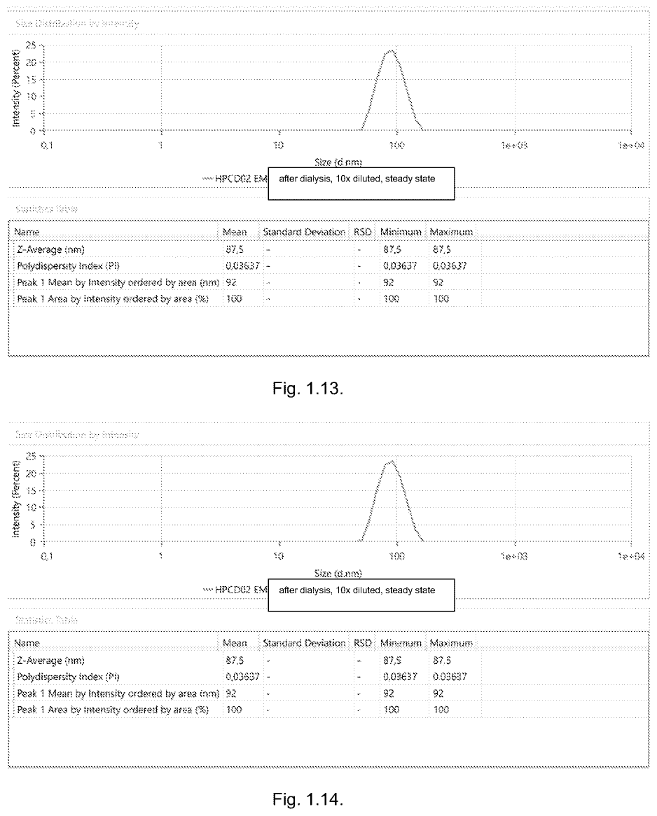

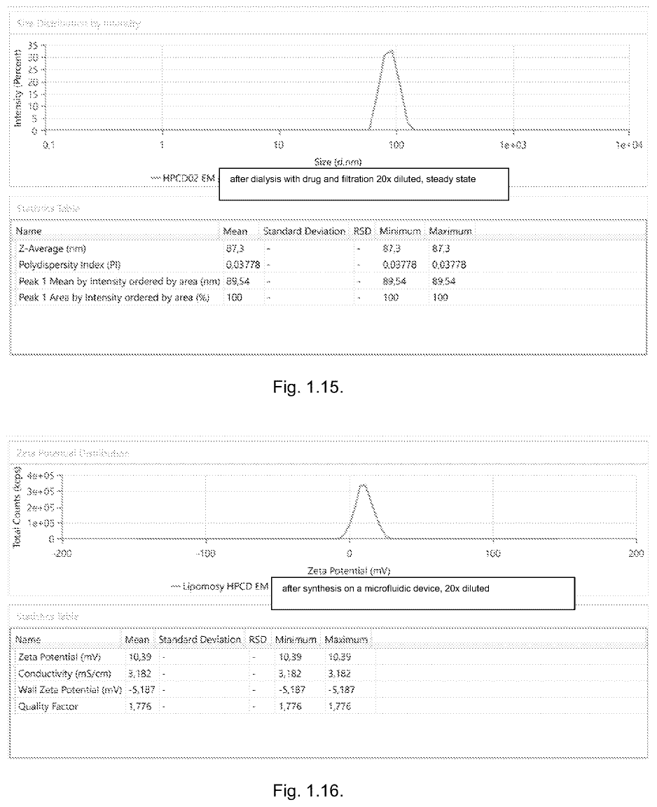

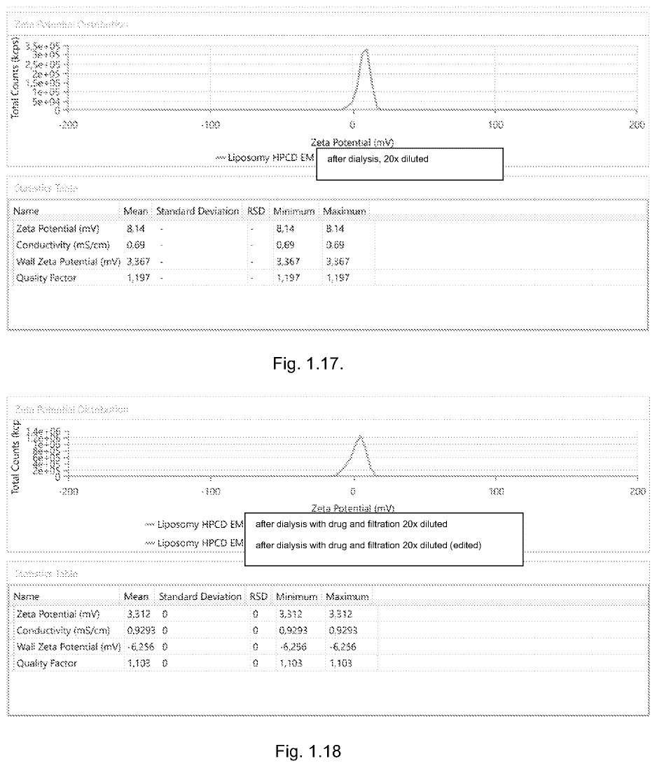

FIG. 1.1 shows an example of the size distribution of hydrodynamic radii of 14 pH EM liposomes after synthesis on a microfluidic device, measured by dynamic light scattering for a sample diluted 10 times;

FIG. 1.2.—Example of the size distribution of hydrodynamic radii of 14 pH EM liposomes after changing the external solution to PBS buffer pH 7.4, measured by dynamic light scattering for a sample diluted 10 times;

FIG. 1.3.—Example of the size distribution of hydrodynamic radii of 14 pH EM liposomes after emetine loading and purification, measured by dynamic light scattering for a sample diluted 10 times;

FIG. 1.4.—Example of Zeta potential distribution for liposomes 14 pH after synthesis on a microfluidic device, measured by dynamic light scattering for a sample diluted 20 times;

FIG. 1.5.—Example of the size distribution of hydrodynamic radii of 14 pH liposomes after replacement of the external solution with PBS buffer pH 7.4, measured by dynamic light scattering for a sample diluted 20 times;

FIG. 1.6.—Example of Zeta potential distribution for liposomes 14 pH after drug loading and final purification, measured by dynamic light scattering for a sample diluted 20 times;

FIG. 1.7.—Example of the size distribution of hydrodynamic radii of 14HSA liposomes after synthesis on a microfluidic device, measured by dynamic light scattering for a sample diluted 10 times;

FIG. 1.8.—Example of the size distribution of hydrodynamic radii of 14HSA liposomes after replacement of the external solution with PBS buffer pH 7.4, measured by dynamic light scattering for a sample diluted 10 times,

FIG. 1.9.—Example of the size distribution of hydrodynamic radii of 14HSA liposomes after emetine loading and purification, measured by dynamic light scattering for a sample diluted 10 times;

FIG. 1.10.—Example Zeta potential distribution for 14 HSA liposomes after synthesis on a microfluidic device, measured by dynamic light scattering for a sample diluted 20 times;

FIG. 1.1.—Example of the size distribution of hydrodynamic radii of 14HSA liposomes after replacement of the external solution with PBS buffer pH 7.4, measured by dynamic light scattering for a sample diluted 20 times;

FIG. 1.12.—Example of Zeta potential distribution for 14 HSA liposomes after drug loading and final purification, measured by dynamic light scattering for a sample diluted 20 times;

FIG. 1.13.—Example of the size distribution of hydrodynamic radii of HPCD02 liposomes after synthesis on a microfluidic device, measured by dynamic light scattering for a sample diluted 10 times;

FIG. 1.14.—Example of the size distribution of hydrodynamic radii of HPCD02 liposomes after replacement of the external solution with PBS buffer pH 7.4, measured by dynamic light scattering for a sample diluted 10 times;

FIG. 1.15.—Example of the size distribution of hydrodynamic radii of HPCD02 liposomes after emetine loading and purification, measured by dynamic light scattering for a sample diluted 10 times;

FIG. 1.16.—Example Zeta potential distribution for HPCD02 liposomes after synthesis on a microfluidic device, measured by dynamic light scattering for a sample diluted 20 times;

FIG. 1.17.—Example of Zeta potential distribution for HPCD02 liposomes after dialysis in PBS buffer pH 7.4 before the drug loading step, measured by dynamic light scattering for a sample diluted 20 times;

FIG. 1.18.—Example of Zeta potential distribution for HPCD02 liposomes after drug loading and final purification, measured by dynamic light scattering for a sample diluted 20 times;

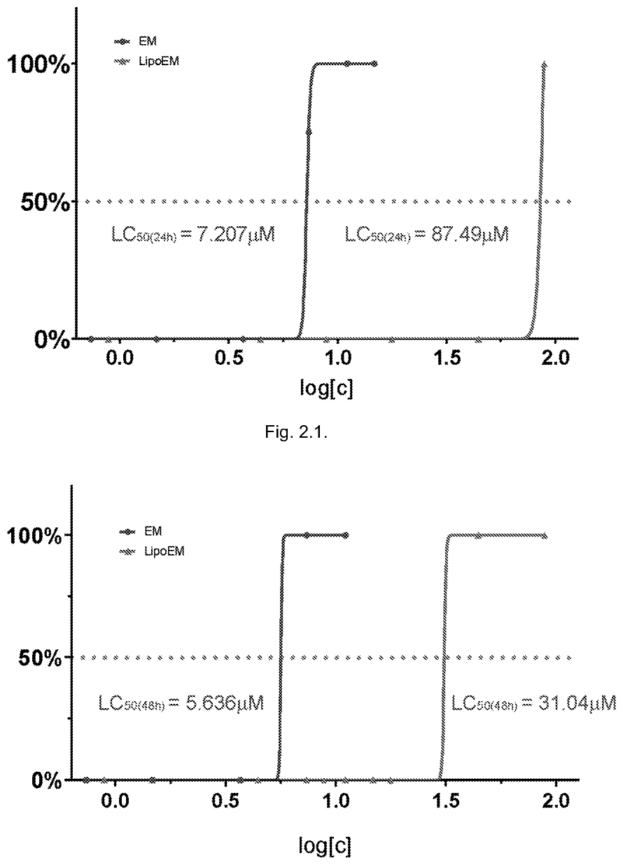

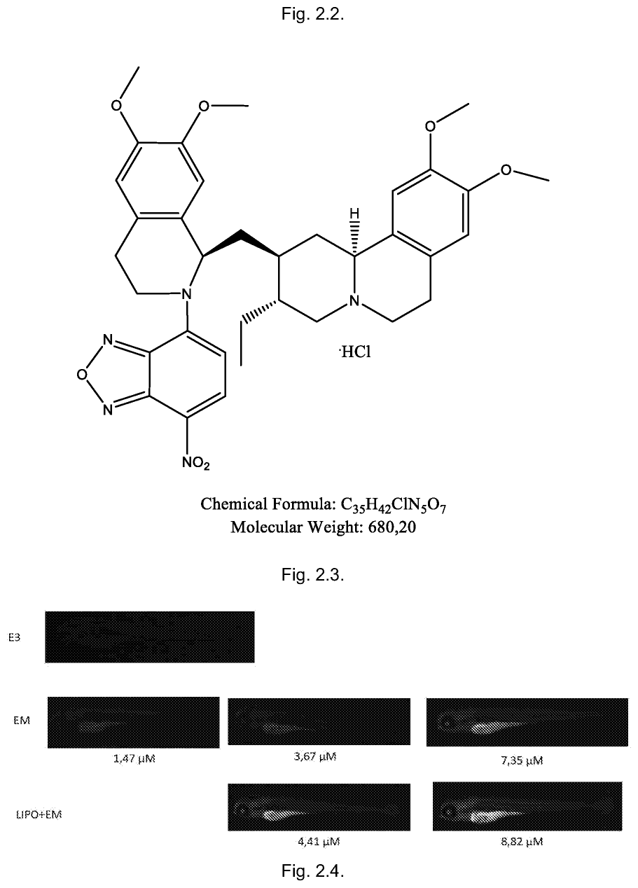

FIG. 2.1.—Comparison of toxicity after 24 hours of exposure to emetine and liposomes with emetine;

FIG. 2.2.—Comparison of toxicity after 48 hours of exposure to emetine and 14 pH EM liposomes with emetine;

FIG. 2.3.—Structure of fluorescently labelled emetine;

FIG. 2.4.—Results of 4 h absorption of emetine and emetine in 15 pH EM liposomes, 2-day-old larvae immersed in: buffer (E3), fluorescently labelled emetine solution (EM) and fluorescently labelled emetine encapsulated in pH-type liposomes. (LIPO+EM);

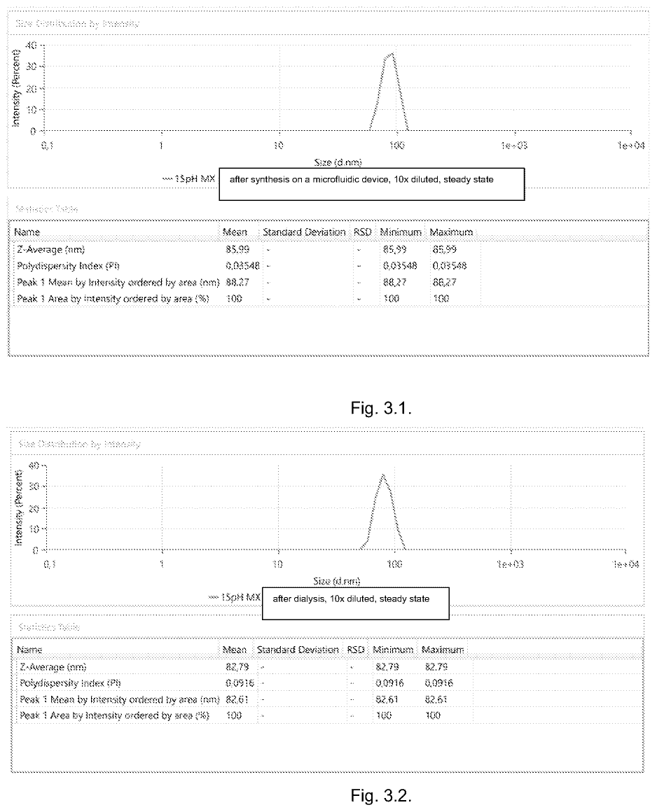

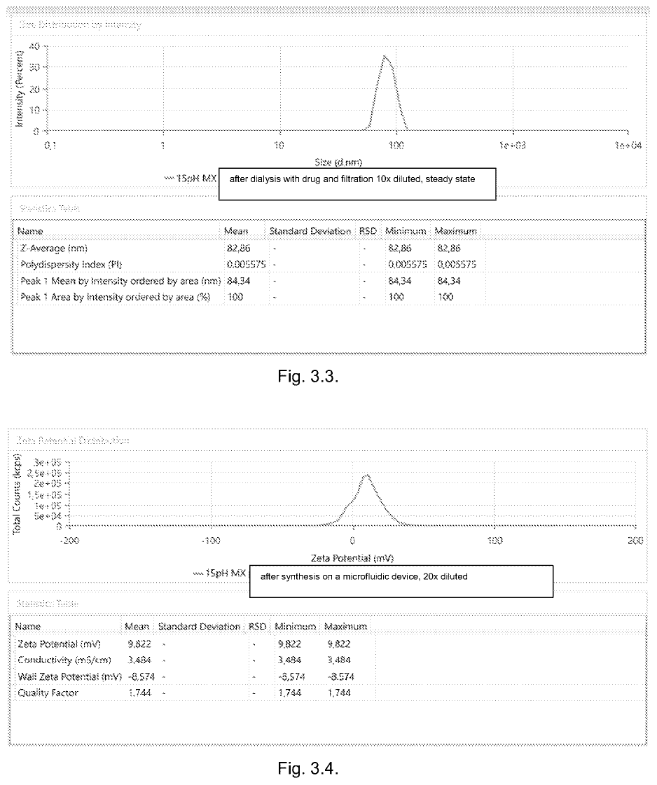

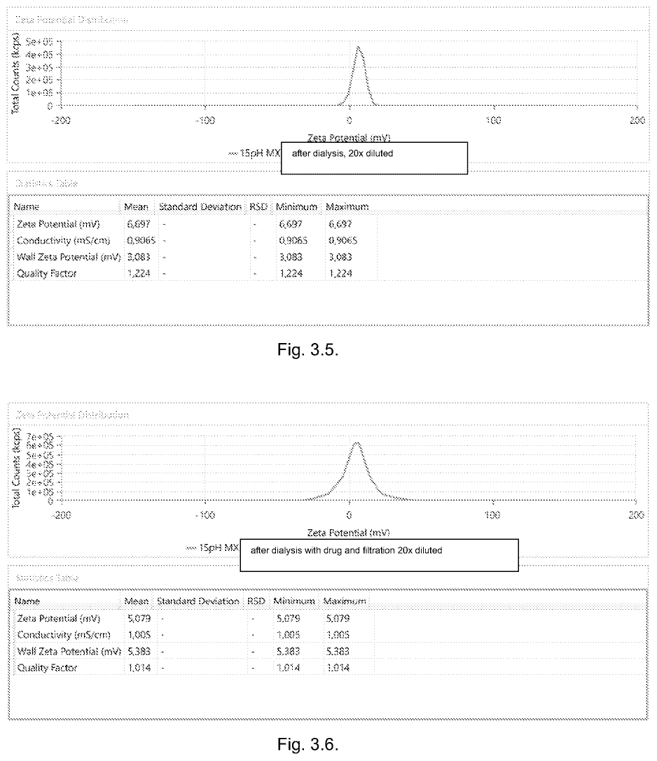

FIG. 3.1.—Example of the size distribution of hydrodynamic radii of 15 pH MX liposomes after synthesis on a microfluidic device, measured by dynamic light scattering for a sample diluted 10 times.

FIG. 3.2.—Example of the size distribution of hydrodynamic radii of 15 pH MX liposomes after replacing the external solution with PBS buffer pH 7.4, measured by dynamic light scattering for a sample diluted 10 times;

FIG. 3.3.—Example of the size distribution of hydrodynamic radii of 15 pH MX liposomes after mitoxantrone loading and purification, measured by dynamic light scattering for a sample diluted 10 times;

FIG. 3.4.—Example Zeta potential distribution for 15 pH MX liposomes after synthesis on a microfluidic device, measured by dynamic light scattering for a sample diluted 20 times;

FIG. 3.5—Example of Zeta potential distribution for 15 pH MX liposomes after dialysis in PBS buffer pH 7.4 before the drug loading step, measured by dynamic light scattering for a sample diluted 20 times;

FIG. 3.6.—Example of Zeta potential distribution for 15 pH MX liposomes after drug loading and final purification, measured by dynamic light scattering for a sample diluted 20 times,

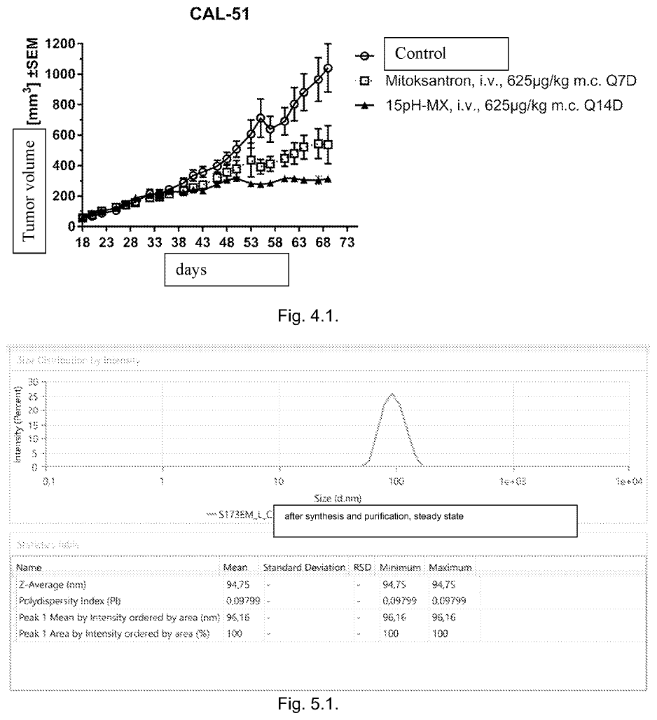

FIG. 4.1—Mean tumour volume since treatment for NOD.Cg-Prkdc strain^{scid} II2rgtm1Sug/JicTac mice: untreated (control), treated with Mitoxantrone at weekly doses of 0.625 mg/kg b.w.(Mitoxantrone) and treated with 15 pH-MX liposomes administered once every fortnight;

FIG. 5.1.—Example distribution of hydrodynamic diameters of S173EM_L_C liposomes after synthesis and purification, measured by dynamic light scattering;

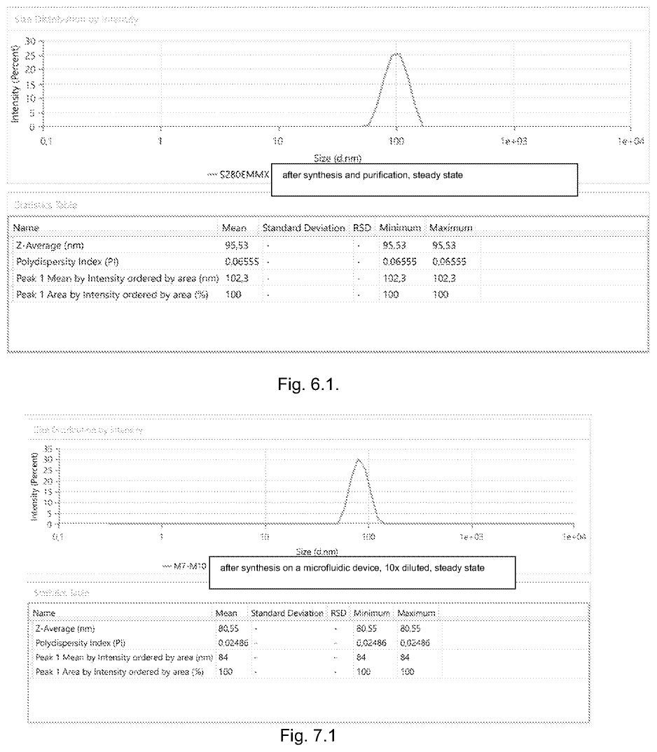

FIG. 6.1.—Example of the hydrodynamic diameter distribution of S280EMMX liposomes after synthesis and purification, measured by dynamic light scattering,

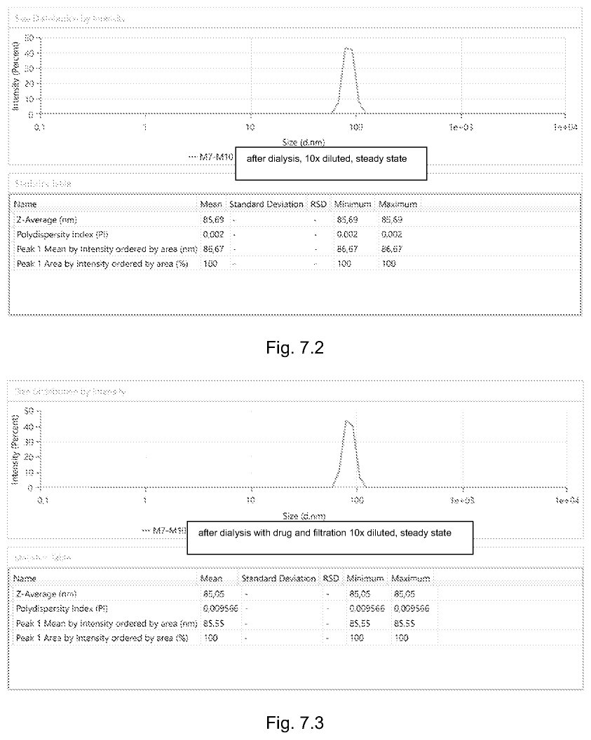

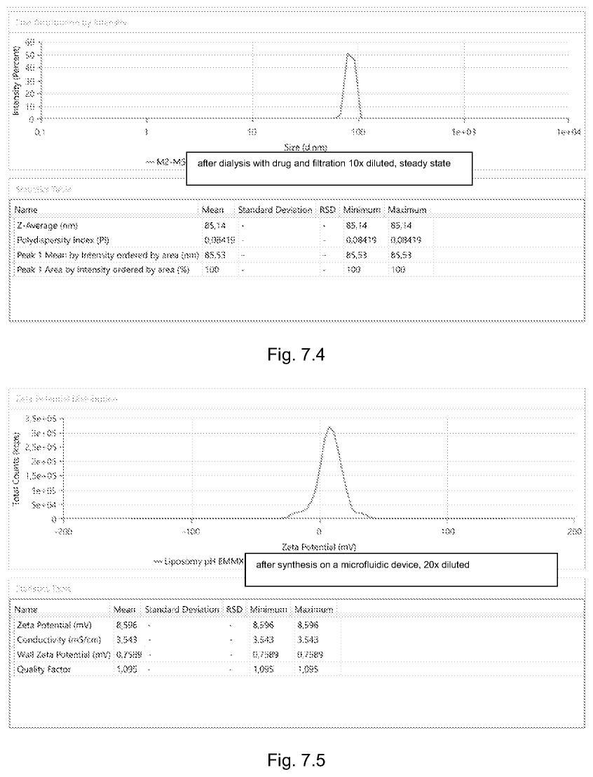

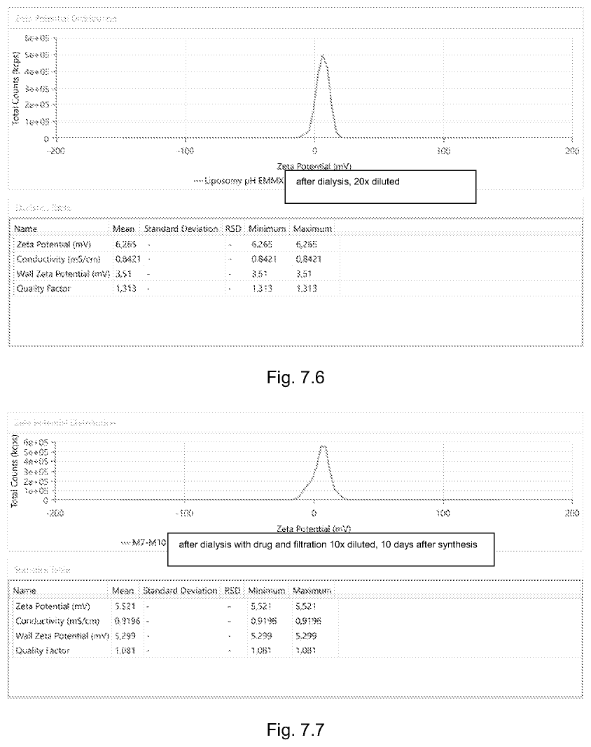

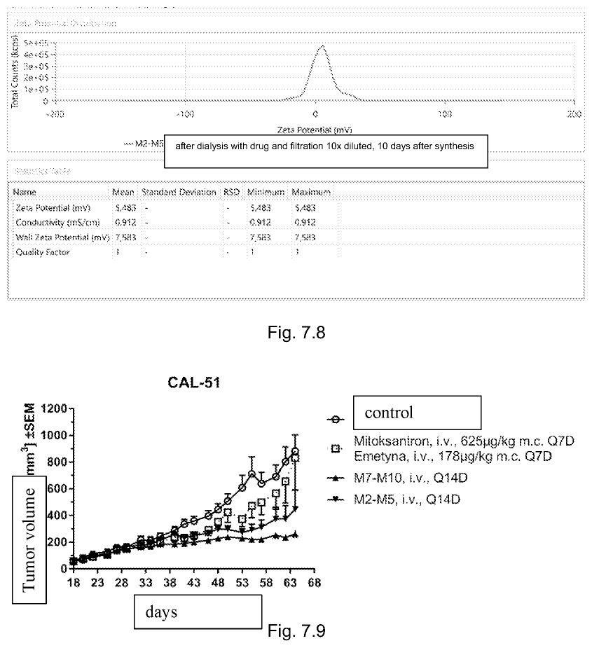

FIG. 7.1.—Example of the size distribution of hydrodynamic radii of M7_M10 liposomes after microfluidic synthesis measured by dynamic light scattering for a sample diluted 10 times;

FIG. 7.2.—Example of the size distribution of hydrodynamic radii of M7_M10 liposomes after dialysis in PBS buffer pH 7.4—before the drug loading step, measured by dynamic light scattering for a sample diluted 10 times;

FIG. 7.3.—Example of the size distribution of hydrodynamic radii of M7 M10 liposomes after drug loading, purification and sterilisation, measured by dynamic light scattering for a sample diluted 10 times;

FIG. 7.4.—Example of the size distribution of hydrodynamic radii of M2_M5 liposomes after drug loading, purification and sterilisation, measured by dynamic light scattering for a sample diluted 10 times,

FIG. 7.5—Example Zeta potential distribution for liposomes M2-M5 and M7-M10 after synthesis on a microfluidic device (Stage 1), measured by dynamic light scattering for a sample diluted 20 times;

FIG. 7.6—Example of Zeta potential distribution for liposomes M2-M5 and M7-M10 after dialysis in PBS buffer pH 7.4 (Stage 11), measured by dynamic light scattering for a sample diluted 20 times;

FIG. 7.7.—Example of Zeta potential distribution for M7-M10 liposomes after drug loading and dialysis purification in PBS buffer pH 7.4 (Stage III), measured by dynamic light scattering for a 20-fold diluted sample;

FIG. 7.8.—Example of Zeta potential distribution for M2-M5 liposomes after drug loading and dialysis purification in PBS buffer pH 7.4 (Stage III), measured by dynamic light scattering for a 20-fold diluted sample;

FIG. 7.9.—plot of mean tumour volume versus time for mice of strain NOD.Cg-Prkdc^{scid} II2rgtmlSug/JicTac: untreated (Control), treated with a mixture of mitoxantrone and emetine administered once a week at doses of 0.625 mg/kg b.w. and emetine 0.179 mg/kg b.w. (Mitoxantrone and Emetine) and treated with M7-M10 liposomes administered once every fortnight;

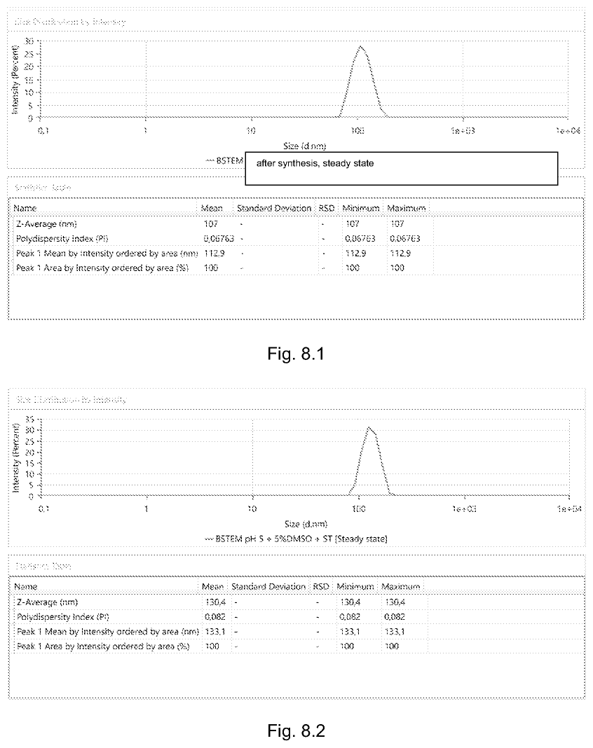

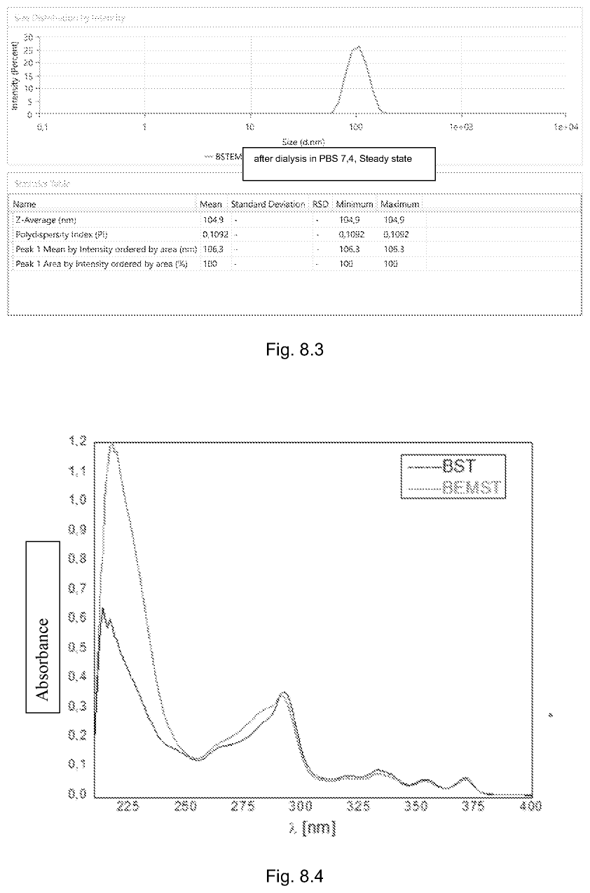

FIG. 8.1—Example of the size distribution of hydrodynamic radii of liposomes after synthesis on a microfluidic device, measured by dynamic light scattering, sample diluted 10 times;

FIG. 8.2.—Example of the size distribution of hydrodynamic radii of liposomes after dialysis in acetate buffer pH 5 and addition of DMSO and staurosporine solution in DMSO, measured by dynamic light scattering for a sample diluted 10 times;

FIG. 8.3—Example of the size distribution of hydrodynamic radii of BSTEM liposomes after final purification measured by dynamic light scattering for a sample diluted 10 times;

FIG. 8.4—UV-Vis spectrum of BST nanoparticles after staurosporine loading and dialysis purification in PBS buffer pH 8.5 (black curve) and staurosporine and emetine loading after dialysis purification in PBS buffer pH 7.4; samples diluted 200× with a solution containing 90% by volume ethanol, 5% by volume 0.3M HCl and 5% by volume PBS buffer pH 7.4;

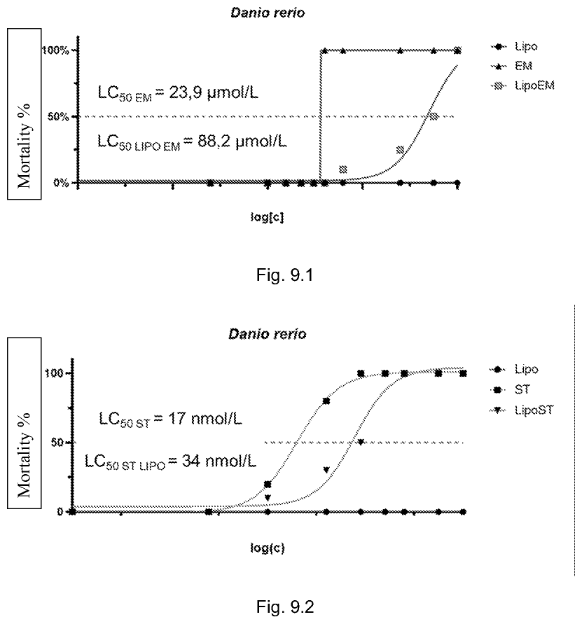

FIG. 9.1.—Comparison of toxicity after 96 hours of exposure of Zebrafish (Danio rerio) to emetine and BEM liposomes with emetine,

FIG. 9.2.—Comparison of toxicity after 96 hours of exposure of Zebrafish (Danio rerio) to staurosporine and BST liposomes with staurosporine.

### EXAMPLE I

Table 1. Lipid Composition of the Organic and Aqueous Phases Used for Liposome Synthesis Along with Microfluidic Conditions.

| | | Liposome | Masses of lipids in the organic phase mg/ml | | | | | | |
|---|---|---|---|---|---|---|---|---|---|
| | | composition (molar | | | DSPE- | | | Flow | Water |
| L.p. | Code | ratios of lipids) | DPPC | Cholesterol | PEG(2000)NH2 | DOPC | DOPG | conditions | phase |
| 1 | pH | DPPC/Cholesterol/ | 70.7 | 18.61 | 10.74 | — | — | FRR 5 ml/min, | 350 mM |
| | | DSPE-PEG(2000)NH2 | | | | | | TFR 9:1 | (NH4) |
| | | (1/0.5/0.04) | | | | | | | 2SO4 |
| 2 | HPCD | DPPC/Cholesterol/ | 70.7 | 18.61 | 10.74 | — | — | FRR 5 ml/min, | 10 |
| | | DSPE-PEG(2000)NH2 | | | | | | TFR 9:1 | mg/ml |
| | | (1/0.5/0.04) | | | | | | | HPCD |
| | | | | | | | | | in |
| | | | | | | | | | 350 mM |
| | | | | | | | | | (NH4) |
| | | | | | | | | | 2SO4 |
| 3 | DOPC | DPPC/DSPE- | 34.1 | 12.83 | 7.41 | 15.7 | — | FRR 5 ml/min, | 350 mM |
| | | PEG(2000)NH2/ | | | | | | TFR 9:1 | (NH4) |
| | | Cholesterol/DOPC | | | | | | | 2SO4 |
| | | (0.7/0.04/0.5/0.3) | | | | | | | or |
| | | | | | | | | | 250 mM |
| | | | | | | | | | (NH)₄₂ |
| | | | | | | | | | SO₄ |
| 4 | DOPC_{—} | DPPC/DSPE- | 19.6 | 6.45 | 3.72 | 5.24 | — | FRR 5 ml/min, | 350 mM |
| | 2 | PEG(2000)NH2/ | | | | | | TFR 9:1 | (NH4) |
| | | Cholesterol/DOPC | | | | | | | 2SO4 |
| | | (0.9/0.04/0.5/0.2) | | | | | | | |
| 5 | DOPC_{—} | DPPC/DSPE- | 22.1 | 6.48 | 3.74 | 2.63 | — | FRR 5 ml/min, | 350 mM |
| | 3 | PEG(2000)NH2/ | | | | | | TFR 9:1 | (NH4) |
| | | Cholesterol/DOPC | | | | | | | 2SO4 |
| | | (0.9/0.04/0.5/0.1) | | | | | | | |
| 6 | DOPC/ | DPPC/DSPE- | 17.0 | 6.42 | 3.7 | 7.82 | — | FRR 5 ml/min, | 10 |
| | HPCD | PEG(2000)NH2/ | | | | | | TFR 9:1 | mg/ml |
| | | Cholesterol/DOPC | | | | | | | HPCD |
| | | (0.7/0.04/0.5/0.3) | | | | | | | in |
| | | | | | | | | | 250 mM |
| | | | | | | | | | (NH4) |
| | | | | | | | | | 2SO4 |
| 7 | DOPG | DPPC/Cholesterol/ | 34.0 | 12.8 | 7.4 | — | 15.8 | FRR 5 ml/min, | 350 mM |
| | | DSPE- | | | | | | TFR 9:1 | (NH4) |
| | | PEG(2000)NH2/DOPG | | | | | | | 2SO4 |
| | | (0.7/0.5/0.04/0.3) | | | | | | | or |
| | | | | | | | | | 250 mM |
| | | | | | | | | | (NH)₄₂ |
| | | | | | | | | | SO₄ |
| 8 | BpH | DPPC/Cholesterol/ | 70.7 | 18.61 | 10.74 | — | — | FRR 103 | 350 mM |
| | | DSPE-PEG(2000)NH2 | | | | | | ml/min TFR | (NH4) |
| | | (1/0.5/0.04) | | | | | | 5:1 | 2SO4 |

### EXAMPLE II

Table 2. Example liposome loading conditions with emetine hydrochloride, together with physicochemical parameters (hydrodynamic diameter—HR, polydispersity index—PDI) and % EE drug encapsulation factors.

| | | | | | | | | Lipid | Drug | | | | | |
|---|---|---|---|---|---|---|---|---|---|---|---|---|---|---|
| | | | | | | | | concen- | addition - | | | | | |
| | | | | Charging conditions | | | | tration | active | | | | | |
| | | | | gradient | | Temper- | | after | charging | | | | | |
| | Liposome | Synthesis | | Initial | Final | ature | Time | synthesis | EM | | | | | % EE |
| L.p. | code | code | Water phase | pH | pH | [C.] | [min] | [mg/ml]. | mg | HR | (SD) | PDI | (SD) | (EM) |
| 1 | pH | 14 pH EM | 350 mM | 5.5 | 7.4 | 60 | 15 | 10 | 1 | 82.05 | 1.74 | 0.014 | 0.012 | 95 |
| | | | (NH)₄₂ SO₄ | | | | | | | | | | | |
| 2 | pH | pH 01 EM m | 350 mM | 5.3 | 7.4 | 60 | 30 | 10 | 0.5 | 85.12 | 0.21 | 0.021 | 0.016 | 97.4 |
| | | | (NH)₄₂ SO₄ | | | | | | | | | | | |
| 3 | pH | pH 02 EM | 350 mM | 2.62 | 8.5 | 60 | 15 | 10 | 0.5 | 80.23 | 0.77 | 0.017 | 0.007 | 96.7 |
| | | | (NH)₄₂ SO₄ | | | | | | | | | | | |
| 4 | pH | pH 03 EM | 350 mM | 2.62 | 7.4 | 60 | 15 | 10 | 0.5 | 86.45 | 1.07 | 0.016 | 0.007 | 98.1 |
| | | | (NH)₄₂ SO₄ | | | | | | | | | | | |
| 5 | pH | 293EM | 350 mM | 5.3 | 8.5 | 60 | 30 | 10.0 | 1.2 | 88.63 | 0.24 | 0.029 | 0.01 | 92.7 |
| | | | (NH4)2SO4 | | | | | | | | | | | |
| 6 | HPCD | HPCD 02 EM | 10 mg/ml | 5.3 | 7.4 | 60 | 30 | 10 | 1 | 87.43 | 0.13 | 0.019 | 0.017 | 95.3 |
| | | | HPCD in | | | | | | | | | | | |
| | | | 350 mM | | | | | | | | | | | |
| | | | (NH4)2SO4 | | | | | | | | | | | |
| 7 | HPCD | HPCD 03 EM | 10 mg/ml | 5.3 | 8.5 | 60 | 30 | 10 | 0.4 | 86.98 | 0.65 | 0.021 | 0.02 | 90 |
| | | | HPCD in | | | | | | | | | | | |
| | | | 350 mM | | | | | | | | | | | |
| | | | (NH4)2SO4 | | | | | | | | | | | |
| 8 | pH/HSA | 14 HSA EM | 350 mM | 5.3 | 7.4 | 60 | 30 | 10 | 1 | 82 | 1.6 | 0.043 | 0.012 | 95 |
| | | | (NH)₄₂ SO₄ | | | | | | | | | | | |
| 9 | DOPC | 167EM | 250 mM | 5.3 | 8.0 | 4 | 1440 | 7.00 | 1.2 | 90.91 | 3.39 | 0.065 | 0.027 | 80.0 |
| | | | (NH)₄₂ SO₄ | | | | | | | | | | | |
| 10 | DOPC | 171EM_G | 250 mM | 5.3 | 9.0 | 45 | 1440 | 7.00 | 1.2 | 89.64 | 3.24 | 0.075 | 0.02 | 79.0 |
| | | | (NH)₄₂ SO₄ | | | | | | | | | | | |
| 11 | DOPC | 173EM_L_C | 250 mM | 5.3 | 8.5 | 4 | 1440 | 7.00 | 1.2 | 95.24 | 1.43 | 0.09 | 0.031 | 71.8 |
| | | | (NH)₄₂ SO₄ | | | | | | | | | | | |
| 12 | DOPC | 173EM_{—} | 250 mM | 5.3 | 8.5 | 4 | 1440 | 7.00 | 0.6 | 88.8 | 0.51 | 0.077 | 0.01 | 77.1 |
| | | L_1/2 | (NH)₄₂ SO₄ | | | | | | | | | | | |
| 13 | DOPC | 173EM_{—} | 250 mM | 5.3 | 8.5 | 22 | 1440 | 7.00 | 0.9 | 95.88 | 3.06 | 0.05 | 0.019 | 86.1 |
| | | P_3/4 | (NH)₄₂ SO₄ | | | | | | | | | | | |
| 14 | DOPC | 173EM_{—} | 250 mM | 5.3 | 8.5 | 22 | 1440 | 7.00 | 1.2 | 86.9 | 3.35 | 0.082 | 0.049 | 96.8 |
| | | P_1/2 | (NH)₄₂ SO₄ | | | | | | | | | | | |
| 15 | DOPC | 181EM_L | 250 mM | 5.3 | 8.0 | 4 | 1440 | 3.5 | 1.2 | 103.5 | 0.71 | 0.018 | 0.023 | 58.0 |
| | | | (NH)₄₂ SO₄ | | | | | | | | | | | |
| 16 | DOPC/HPCD | 201 EM CD | 250 mM | 5.3 | 9.0 | 60 | 60 | 7 | 1.3 | 105.2 | | 0.002 | | 92.7 |
| | | (1:3) C_G | (NH)₄₂ SO₄ | | | | | | | | | | | |
| 17 | DOPC/HPCD | 201 EM CD | 250 mM | 5.3 | 9.0 | 4 | 1440 | 7 | 1.3 | 105.7 | | 0.006 | | 93 |
| | | (1:3) C_L | (NH)₄₂ SO₄ | | | | | | | | | | | |
| 18 | DOPG | S264EM | 250 mM | 5.3 | 8.5 | 4 | 1440 | 7 | 1.2 | 89.72 | 1.23 | 0.052 | 0.009 | 95.2 |
| | | | (NH)₄₂ SO₄ | | | | | | | | | | | |
| 19 | DOPG | S273EM | 250 mM | 5.3 | 8.5 | 60 | 15 | 7 | 1.2 | 89.05 | 0.58 | 0.036 | 0.029 | 64.0 |
| | | | (NH)₄₂ SO₄ | | | | | | | | | | | |
| 20 | DOPG | S274EM | 250 mM | 5.3 | 8.5 | 60 | 30 | 7 | 1.2 | 87.33 | 0.86 | 0.048 | 0.011 | 82.7 |
| | | | (NH)₄₂ SO₄ | | | | | | | | | | | |
| 21 | DOPG | 294EM | 350 mM | 5.3 | 8.5 | 60 | 30 | 7 | 1.2 | 84.85 | 1.96 | 0.115 | 0.01 | 97.6 |
| | | | (NH)₄₂ SO₄ | | | | | | | | | | | |
| 22 | BpH | BES | 350 mM | 5.3 | 7.4 | 4 | 1440 | 10 | 1 | 108.9 | 1.23 | 0.12 | 0.026 | 96.0 |
| | | | (NH)₄₂ SO₄ | | | | | | | | | | | |

### EXAMPLE III

Table 3. Example loading conditions of liposomes with emetine hydrochloride and mitoxantrone dihydrochloride, together with physicochemical parameters (hydrodynamic diameter—HR, polydispersity index—PDI) and % EE drug encapsulation factors.

| | | | Charging conditions | | | | | | | |
|---|---|---|---|---|---|---|---|---|---|---|
| | | | | | T | | Time | T | Time | |
| | | | | | [° C.] | | [min] | [C.] | [min] | Drug addition - active charging |
| | Liposome | Synthesis | Initial | Final | for | | for | for | for | EM |
| I.P. | code | code | pH | pH | EM | Charging | EM | MX | MX | mg/ml |
| 1 | pH | 6 pH | 5.5 | 7.4 | 60 | Total | 15 | 60 | 15 | 0.24 |
| | | EMMX | | | | | | | | |
| 2 | pH | pH 01 | 5.5 | 7.4 | 60 | Total | 30 | 60 | 30 | 0.24 |
| | | EMMX | | | | | | | | |
| 3 | pH | pH 02 | 2.62 | 8.5 | 60 | Total | 15 | 60 | 15 | 0.24 |
| | | EMMX | | | | | | | | |
| 4 | pH | pH 03 | 2.62 | 7.4 | 60 | Total | 15 | 60 | 15 | 0.24 |
| | | EMMX | | | | | | | | |
| 6 | pH | S258EMMX | 5.3 | 8.5 | 60 | Sequential | 60 | 4 | 4320 | 1.2 |
| 7 | pH | S259EMMX | 5.3 | 8.5 | 60 | Sequential | 15 | 60 | 60 | 1.2 |
| 8 | pH | S260EMMX | 5.3 | 8.5 | 60 | Sequential | 30 | 60 | 60 | 1.2 |
| 9 | pH | S261EMMX | 5.3 | 8.5 | 60 | Sequential | 60 | 60 | 60 | 1.2 |
| 10 | pH | S270EMMX | 5.3 | 8.5 | 60 | Total | 15 | 60 | 15 | 0.6 |
| 11 | pH | S272EMMX | 5.3 | 8.5 | 60 | Total | 15 | 60 | 15 | 0.6 |
| 12 | pH | S281EMMX | 5.3 | 8.5 | 60 | Total | 15 | 60 | 15 | 1.2 |
| 13 | pH | S282EMMX | 5.3 | 8.5 | 60 | Total | 15 | 60 | 15 | 1.2 |
| 14 | HPCD | HPCD | 5.3 | 7.4 | 60 | Total | 30 | 60 | 30 | 0.24 |
| | | 01 | | | | | | | | |
| | | EMMX | | | | | | | | |
| 15 | HPCD | HPCD | 5.3 | 8.5 | 60 | Total | 30 | 60 | 30 | 0.24 |
| | | 03 | | | | | | | | |
| | | EMMX | | | | | | | | |
| 16 | HPCD | 3HPCD | 5.5 | 7.4 | 60 | Total | 15 | 60 | 15 | 0.24 |
| | | EMMX | | | | | | | | |
| 17 | pH/ | 7HSA | 5.5 | 7.4 | 60 | Total | 15 | 60 | 15 | 0.24 |
| | HSA | EMMX | | | | | | | | |
| 18 | pH/HSA | M2-M5 | 5.5 | 7.4 | 60 | Total | 15 | 60 | 15 | 0.24 |
| 19 | pH | M7-M10 | 5.5 | 7.4 | 60 | Total | 15 | 60 | 15 | 0.24 |
| 20 | | | | | | | | | | |
| 21 | DOPG | S276EMMX | 5.3 | 8.5 | 60 | Total | 30 | 60 | 30 | 1.2 |
| 22 | DOPG | S280EMMX | 5.3 | 8.5 | 60 | Total | 30 | 60 | 30 | 1.2 |

| | | Physico-chemical parameters | | | | | | |
|---|---|---|---|---|---|---|---|---|
| | Drug addition - active charging | | | | | % EE | % (EE) | Ratio |
| I.P. | MX mg/ml | HR | (SD) | PDI | (SD) | (EM) | MAX | MX/EM |
| 1 | 0.9 | 87.7 | 1.18 | 0.037 | 0.02 | 92 | 96 | 3.91 |
| 2 | 0.9 | 91.36 | 1.32 | 0.076 | 0.016 | 87.2 | 98.9 | 4.25 |
| 3 | 0.9 | 88.3 | 0.026 | 0.019 | 0.021 | 77.1 | 99.4 | 4.83 |
| 4 | 0.9 | 99.42 | 4.65 | 0.134 | 0.006 | 64.4 | 99.4 | 5.79 |
| 6 | 1.5 | 86.24 | 0.34 | 0.016 | 0.005 | 83.7 | 26.3 | 0.39 |
| 7 | 1.5 | 93.68 | 0.92 | 0.016 | 0.005 | 76.2 | 94.4 | 1.55 |
| 8 | 1.5 | 87.62 | 1.26 | 0.063 | 0.005 | 82.6 | 94.4 | 1.43 |
| 9 | 1.5 | 89.68 | 3.21 | 0.01 | 0.01 | 88.3 | 95.7 | 1.35 |
| 10 | 0.8 | 121 | 1.93 | 0.2 | 0.017 | 59.1 | 99.3 | 2.24 |
| 11 | 0.6 | 87.67 | 1.68 | 0.031 | 0.014 | 81.7 | 99.3 | 1.22 |
| 12 | 0.6 | 85.51 | 0.2 | 0.021 | 0.018 | 89.6 | 99.2 | 0.55 |
| 13 | 0.9 | 86.09 | 0.4 | 0.011 | 0.008 | 78.2 | 98.9 | 0.95 |
| 14 | 0.9 | 102.5 | 1.47 | 0.098 | 0.018 | 91 | 99.6 | 4.10 |
| 15 | 0.9 | 120.9 | 14.77 | 0.169 | 0.018 | 77 | 99 | 4.82 |
| 16 | 0.9 | 85.74 | 1.14 | 0.088 | 0.013 | 89 | 89 | 3.75 |
| 17 | 0.9 | 83.06 | 0.46 | 0.044 | 0.027 | | 95 | 3.93 |
| 18 | 0.9 | 86.07 | 0.74 | 0.03 | 0.018 | 90 | 97 | 4.04 |
| 19 | 0.9 | 85.27 | 0.58 | 0.02 | 0.016 | 98 | 100 | 3.83 |
| 20 | | | | | | | | |
| 21 | 1.2 | 97.23 | 3.45 | 0.111 | 0.015 | 591 | 99.1 | 1.68 |
| 22 | 0.6 | 98.14 | 4.06 | 0.062 | 0.004 | 82.3 | 98.9 | 0.60 |

### EXAMPLE IV

Table 4. Example loading conditions of mitoxantrone dihydrochloride liposomes together with physicochemical parameters (hydrodynamic diameter—HR, polydispersity index—PDI) and % EE drug encapsulation factors.

| | | | Charging conditions | | | | Lipid | | | | | | |
|---|---|---|---|---|---|---|---|---|---|---|---|---|---|
| | | | | | | | concentration | | | | | | |
| | | | gradient | | | | after | MX | | | | | |
| | Sample | Liposome | Initial | Final | Temperature | Time | synthesis | mg/ | | | | | % EE |
| L, p, | code | code | pH | pH | [C.] | [min] | [mg/ml]. | ml | HR | (SD) | PDI | (SD) | MX |
| 1 | 15 pH | pH | 5.5 | 7.4 | 60 | 15 | 10.00 | 0.90 | 83.82 | 21551.00 | 0.02 | 0.02 | 99.9 |
| | MX | | | | | | | | | | | | |
| 2 | pH 01 | pH | 5.3 | 7.4 | 60 | 30 | 10.00 | 0.90 | 104.8 | 0.61 | 0.19 | 0.00 | 99.4 |
| | MX m | | | | | | | | | | | | |
| 3 | pH 02 | pH | 2.6 | 8.5 | 60 | 15 | 10.00 | 0.90 | 81.3 | 0.27 | 0.01 | 0.01 | 99.6 |
| | MX | | | | | | | | | | | | |
| 4 | pH 03 | pH | 2.6 | 7.4 | 60 | 15 | 10.00 | 0.90 | 91.1 | 0.29 | 0.14 | 0.01 | 99.5 |
| | MX | | | | | | | | | | | | |
| 5 | 293 MX | pH | 5.3 | 8.5 | 60 | 30 | 10.00 | 1.20 | | | | | 99.0 |
| 6 | HPCD | HPCD | 5.3 | 7.4 | 60 | 30 | 10.00 | 0.90 | 90.6 | 0.42 | 0.03 | 0.02 | 99.6 |
| | 02 MX | | | | | | | | | | | | |
| 7 | HPCD | HPCD | 5.3 | 8.5 | 60 | 30 | 10.00 | 0.90 | 92.0 | 0.56 | 0.01 | 0.01 | 99.9 |
| | 03 MX | | | | | | | | | | | | |
| 8 | 15 HSA | pH/HSA | 5.3 | 7.4 | 60 | 15 | 10.00 | 0.90 | 83.1 | 1.61 | 0.08 | 0.03 | 94.7 |
| | MX | | | | | | | | | | | | |
| 9 | 167MX | DOPC_{—} | 5.3 | 8.0 | 60 | 30 | 7.00 | 1.20 | 84.9 | 1.23 | 0.07 | 0.00 | 74.9 |
| | | 1 | | | | | | | | | | | |
| 10 | 171MX_{—} | DOPC_{—} | 5.3 | 9.0 | 60 | 30 | 7.00 | 1.20 | 86.9 | 0.28 | 0.12 | 0.04 | 95.5 |
| | G | 1 | | | | | | | | | | | |
| 11 | 180MX_{—} | DOPC_{—} | 5.3 | 8.0 | 45 | 15 | 3.50 | 1.20 | 124.8 | 0.98 | 0.07 | 0.02 | 93.7 |
| | G | 3 | | | | | | | | | | | |
| 12 | 181MX_{—} | DOPC_{—} | 5.3 | 8.0 | 45 | 30 | 3.50 | 1.20 | 100.9 | 0.45 | 0.10 | 0.09 | 85.4 |
| | G | 3 | | | | | | | | | | | |
| 13 | 294MX | DOPG | 5.3 | 8.5 | 60 | 30 | 7.00 | 1.20 | 88.3 | 1.11 | 0.06 | 0.02 | 99.6 |

### EXAMPLE V

Table 5. Example loading conditions of liposomes with emetine hydrochloride and/or staurosporine together with physico-chemical parameters (hydrodynamic diameter—HR, polydispersity index—PDI) and % EE drug encapsulation factors.

| | | | drug addition - | | | | | | | | |
|---|---|---|---|---|---|---|---|---|---|---|---|
| | | | active charging | | | | | | | | |
| | Synthesis | Liposome | EM | ST | | | | | % EE | %(EE) | Ratio |
| L.p. | code | code | mg/ml | mg/ml | HR | (SD) | PDI | (SD) | (EM) | ST | ST/EM |
| 1 | ST25 | pH | — | 1 | 77.7 | 0.18 | 0.011 | 0.006 | — | 79 | — |
| 2 | ST40 | pH | — | 1.6 | 80.4 | 0.095 | 0.008 | 0.004 | — | 73 | — |
| 3 | ST02EM25 | pH | 1 | 0.1 | 83.4 | 1.34 | 0.023 | 0.008 | 91 | 90 | 0.17 |
| 4 | ST10EM25 | pH | 1 | 0.4 | 82.7 | 0.89 | 0.012 | 0.004 | 83 | 78 | 4.6 |
| 5 | BST | BpH | | 0.8 | 110.0 | 0.78 | 0.113 | 0.011 | — | 80 | — |
| 6 | BSTEM | BpH | 1 | 0.8 | 104.9 | 1.12 | 0.109 | 0.024 | 69 | 64 | 0.88 |

### EXAMPLE VI

DPPC/Cholesterol/DSPE-PEG-NH2 Liposomes Loaded with Emetin, pH, HPCD and HSA Variants.

A lipid solution was prepared by weighing out 212 mg of DPPC, 55.8 mg of cholesterol and 32.1 mg of DSPE-PEG(2000)NH2 and dissolving in 3 ml of ethanol. The total was then heated for 15 minutes at 60° C. 1.85 g (NH)₄₂ SO₄, was weighed in sterile urea and dissolved in 40 mL of deionised water. The pH of the solution was set at 5.3 by titration with 0.3 M HCl and 0.2M NaOH solutions. The solution was filtered into a sterile urethra using sterile syringe filters with a hydrophilic PTFE membrane with a pore diameter of 0.22 μm and labelled ‘Solution 1’. Subsequently, 100 mg of hydroxypropyl-β-cyclodextrin (HPCD) was weighed and dissolved in 10 ml—labelled ‘Solution 2’.

Three liposome syntheses were carried out in a NanoAssemblr IGNITE microfluidic Device™ with an NxGen cartridge setting the following flow conditions: aqueous to organic phase volume ratio 9:1; total flow rate 5 ml/min, final reaction mixture volume 8.1 ml; initial loss 0.85 ml; final loss 0.05 ml.

Synthesis I (Liposomes pH) and II (Liposomes pH/HSA): 0.9 ml of lipid solution was placed in a microfluidic device in a 1 ml syringe. 8.1 ml of Solution 1 was taken into a 10 ml syringe and synthesis was started on the microfluidic device. The hydrodynamic diameter and PDI of the resulting liposomes were then measured.

Synthesis III (HPCD liposomes): 0.9 ml of a solution of lipids in ethanol was placed in a microfluidic device in a 1 ml syringe. 8.1 ml of Solution 2 was taken into a 10 ml syringe and the synthesis was started on the microfluidic device. The hydrodynamic diameter and PDI of the resulting liposomes were then measured.

The solutions with syntheses I-III were then dialysed in PBS buffer, pH 7.4. A 5-fold PBS buffer concentrate pH 7.4 was prepared by dissolving 20 g NaCl, 3.6 g Na₂ HPO₄ (anhydrous), 0.775 g NaH₂ PO×2H₄₂ O (dihydrate), 0.5 g KCl in 0.5 L deionised water (milliQ). The mixture was stirred on a magnetic stirrer for 30 min. Then, 400 ml of PBS buffer concentrate (5×) was measured using a measuring cylinder and transferred to a beaker. Subsequently, 1600 ml of distilled water was added. 0.1 M NaOH was added to the buffer until a pH of 7.4 was obtained (acceptable pH range 7.38-7.42). Three tubular dialysis membranes with MWCO pore diameter of 100 kD and volume of 10 ml each were conditioned for 20 min each time in solutions of, successively: 10% ethanol, deionised water and PBS buffer, pH 7.4. The conditioning solutions were previously filtered through sterile syringe filters with a hydrophilic PTFE membrane with a pore diameter of 0.22 μm.

The pH, HSA, HPCD liposomes synthesised in syntheses I-III were then placed in the membranes. Mixing elements and membranes with liposomes were placed in three glass bottles containing PBS pH 7.4. The volume of PBS buffer was chosen so that the buffer completely surrounded the liposomes in the membrane. The bottles were placed on magnetic stirrers in a refrigerator (4° C.). After 3 hours, the buffer was replaced and dialysis continued for 12 hours.

Subsequently, an emetine hydrochloride stock solution of 50 mg/ml was prepared. Then, 20 uL of emetine hydrochloride stock solution was added to the liposomes from Synthesis I_III for each 1 ml of liposomes and mixed. The liposomes were heated in a 60° C. water bath for 15 minutes. After removal from the water bath, the liposomes were allowed to cool for 30 minutes. The vessel with liposomes was protected from light.

The pH and HPCD liposomes were filtered through sterile syringe filters with a hydrophobic PTFE membrane with a pore diameter of 0.45 μm. 0.5 ml of each sample was separated on a chromatography column and % EE drug encapsulation rates were calculated by determining free drug and drug bound in the liposomes using UV-Vis spectrophotometry against a calibration curve. The rest of the liposomes were dialysed in PBS buffer pH 7.4 for 16 h changing the dialysis solution three times after 1 h, 3 h and 15 h. The hydrodynamic radius, polydispersion coefficient and zeta potential of the liposomes at each synthesis step were investigated using dynamic light scattering. The results are summarised in Tables: 6.1, and 6.3 and in Figs: FIG. 1.1-FIG. 1.6 and FIG. 1.13-FIG. 1.18 for pH and HPCD liposomes, respectively.

Liposomes from synthesis II (HSA liposomes) were mixed with a 10% aqueous solution of human albumin in a 3:1 ratio and then incubated at 25° C. for 1 h. 0.5 ml of the sample was separated on a chromatography column and the % EE drug encapsulation factor was calculated by determining the free drug and the drug bound in the liposome using UV-Vis spectrophotometry against a calibration curve. The rest of the liposomes were dialysed in PBS buffer pH 7.4 for 16 h changing the dialysis solution three times after 1 h, 3 h and 15 h. The hydrodynamic radius, polydispersion coefficient and zeta potential of the liposomes at each synthesis step were investigated using dynamic light scattering. The results are summarised in Tables: 6.2 and FIG. 1.7-FIG. 1.12. The values of the % EE coefficients are summarised in Table 6.4.

TABLE 6.1
Physical parameters (hydrodynamic diameter, polydispersion
coefficient and Zeta potential) of 14 pH EM liposomes
after successive synthesis steps.

| | Hydrodynamic | | Zeta |
|---|---|---|---|
| | diameter | | potential |
| Synthesis stage | [nm] | PDI | [mV] |
| Stage I - Microfluid | 89.76 ± 1.47 | 0.02 ± 0.011 | 7.83 ± 0.27 |
| synthesis | | | |
| Step II - change of | 82.36 ± 1.05 | 0.012 ± 0.012 | 7.20 ± 0.26 |
| buffer to PBS pH 7.4 | | | |
| Stage III - drug loading | 82.05 ± 1.74 | 0.014 ± 0.012 | 5.09 ± 0.78 |

The absence of significant changes in hydrodynamic diameters and PDI coefficients indicates the stability of the liposomes during synthesis and the absence of carrier aggregation. The similar Zeta potential value of the liposomes before and after loading with positively charged emetine hydrochloride indicates that the drug is loaded inside the liposomes and not deposited in the outer polymer layer of the carrier.

TABLE 6.2
Physical parameters (hydrodynamic diameter, polydispersion
coefficient and zeta potential) of 14HSAEM liposomes
after successive synthesis steps.

| | Hydrodynamic | | Zeta |
|---|---|---|---|
| | diameter | | potential |
| The synthesis stage | [nm] | PDI | [mV] |
| Stage I - Microfluid | 86.82 ± 1.51 | 0.068 ± 0.083 | 8.40 ± 0.43 |
| synthesis | | | |
| Step II - change of | 79.95 ± 1.47 | 0.027 ± 0.017 | 7.60 ± 0.26 |
| buffer to PBS pH 7.4 | | | |
| Stage III - drug loading | 82 ± 1.6 | 0.043 ± 0.012 | 4.15 ± 0.1 |

The absence of significant changes in hydrodynamic diameters and PDI coefficients indicates the stability of the liposomes during synthesis and the absence of carrier aggregation. The similar Zeta potential value of the liposomes before and after loading with positively charged emetine hydrochloride indicates that the drug is loaded inside the liposomes and not deposited in the outer polymer layer of the carrier.

TABLE 6.3
Physical parameters (hydrodynamic diameter, polydispersion
coefficient and zeta potential) of HPCDEM liposomes
after successive synthesis steps.

| | Hydrodynamic | | Zeta |
|---|---|---|---|
| | diameter | | potential |
| Synthesis stage | [nm] | PDI | [mV] |
| Stage I - Microfluid | 116.3 ± 1.2 | 0.063 ± 0.001 | 10.4 ± 0.21 |
| synthesis | | | |
| Step II - change of | 87.8 ± 1.20 | 0.046 ± 0.032 | 7.90 ± 0.26 |
| buffer to PBS pH 7.4 | | | |
| Stage III - drug loading | 85.74 ± 1.14 | 0.088 ± 0.013 | 3.32 ± 0.38 |

The absence of significant changes in hydrodynamic diameters and PDI coefficients indicates the stability of the liposomes during synthesis and the absence of carrier aggregation. The similar Zeta potential value of the liposomes before and after loading with positively charged emetine hydrochloride indicates that the drug is loaded inside the liposomes and not deposited in the outer polymer layer of the carrier.

TABLE 6.3
Summary of % EE encapsulation rates for
pH, HSA and HPCD series liposomes.

| | 14 pH EM | 14 HSA EM | HPCD02 |
|---|---|---|---|
| % EE | 95.0% | 95.0% | 95.3% |

In addition, the maximum loading of emetine hydrochloride into pH and HPCD liposomes was checked by adding twice and four times higher concentrations of emetine hydrochloride during the drug loading process. The results are shown in Table 6.4.

TABLE 6.4
Encapsulation rates of emetine hydrochloride (EM) into
pH and HPCD liposomes and drug to lipid ratios (D/L
drug to lipid ratio) added for synthesis and obtained
after loading and purification of liposomes.

| | | | Added to | |
|---|---|---|---|---|
| | EM additive for | | D/L | Uploaded |
| Sample name | synthesis mg/ml | % EE | synthesis | D/L |
| pH 1 mg EM | 1 | 95.4 | 0.1 | 0.10 |
| pH 2 mg EM | 2 | 69.96 | 0.2 | 0.14 |
| pH 4 mg EM | 4 | 58.16 | 0.4 | 0.23 |
| HPCD 1 mg EM | 1 | 95.3 | 0.1 | 0.10 |
| HPCD 4 mg EM | 4 | 54.88 | 0.4 | 0.22 |

The high % EE values even for liposomes with 4 mg of drug added per 10 mg of liposomes are indicative of the high loading capacity of this type of liposomes with emetine hydrochloride. Previous reports in the literature show liposome carriers capable of binding only 55% of emetine hydrochloride using a drug to lipid D/L ratio added to the synthesis of 0.1 (https://doi.org/10.1016/j.ejpb.2014.04.002). In the case of HPCD and pH liposomes, a fourfold higher carrier loading of emetine hydrochloride was achieved.

Subsequently, the degree of drug leakage from 14 pH EM, 14HSA EM and HPCD02 EM liposomes was investigated in the presence of albumin:
bovine BSA—albumin used in vitro as an additive to cell medium
rat RSA—the albumin available in rat blood during in vivo studies.
Human HSA—a protein found in approximately 5% of human blood

The results are summarised in Table 6.5.

% of released EM from liposomes
(24 h incubation. 37° C., 5% protein)
[%]

| Sample code | BSA | RSA | HSA |
|---|---|---|---|
| 14 pH EM | 45.4 | 17.1 | 22.27 |
| 14HSA EM | 20.4 | 11.8 | 10.2 |
| HPCD02 EM | 13.6 | 22.7 | 8.6 |

Both the presence of hydroxypropyl-β-cyclodextrin and HSA in the liposome structure limit the rapid ejection of drug from within the liposomes in the presence of bovine serum albumin (BSA). All carriers show high stability in the presence of rat albumin (RSA) and human albumin (HSA). No sudden leakage of drug from the carrier was observed and the drug release rate was classified as less than 23%, indicating that these liposomes can be used as carriers for emetine with extended drug release.

### EXAMPLE VII

### Comparison of Toxicity of Emetine Alone and Emetine in 14 pH EM Liposomes—In Vivo Studies

In order to compare the toxicity of emetine and 14 pH EM liposomes with emetine, an acute toxicity test was conducted on Zebrafish (Danio rerio). The test was designed to determine the acute or lethal toxicity of the formulations on the embryonic stages of the fish. Briefly, Zebrafish 2 dpf larvae were treated with emetine and 14 pH EM liposomes with emetine for 24 and 48 hours at concentrations ranging from 0.73 μM to 125 μM calculated for emetine. A static system was used in the experiment, as changes in solution concentrations did not exceed a range of 20% of the nominal concentrations. The water for embryo culture and solution preparation was embryo culture medium (E3 solution: 5 mmol/L NaCl, 0.17 mmol/L KCl, 0.33 mmol/L CaCl₂ and 0.33 mmol/L MgSO₄, containing no methylene blue, and with a pH value of approximately 7.2). The experiment was carried out in 24-well plates, 5 embryos per well, 10 per group. The plate was covered and kept in an incubator set at 28±0.5° C. with a light/dark period of 12/12 hours. During the exposure period, larvae were tracked every 24 hours and four visual observations, including coagulation of fertilised eggs, lack of somite formation, lack of separation of tail bud from yolk sac and lack of heartbeat, were recorded as indicators of mortality.

At the end of the exposure period (24 and 48 h), acute toxicity was determined based on a positive result from any of the four recorded visual observations and the LC50 was calculated. FIG. 19 shows a comparison of toxicity after 24 h exposure to emetine and emetine liposomes. The half maximum lethal concentration (LC50) calculated for emetine was 7.207 μM, while for emetine encapsulated in 14 pH EM liposomes it was 87.49 μM or 12 times higher.

The results are shown in FIG. 2.1 and FIG. 2.2. In the sample illustrated in FIG. 2.1, the half maximum lethal concentration (LC50) calculated for emetine was 7,207 μM, whereas for emetine encapsulated in 14 pH EM liposomes it was 87.49 μM or 12 times higher. In contrast, in the sample illustrated in FIG. 2.2, the half maximal lethal concentration (LC50) calculated for emetine was 5.636 μM, while for emetine encapsulated in liposomes it was 31.04 μM or 5.5 times higher.

### EXAMPLE VIII

### Comparison of Absorption of Emetine Alone and Emetine in 14 pH EM Liposomes—In Vivo Studies

To compare the uptake of emetine and pH-type liposomes with emetine, Zebrafish larvae were exposed to fluorescently labelled free emetine and fluorescently labelled emetine encapsulated in pH-type liposomes (synthesis identical to that of 14pHEM) for 4 and 48 h in a concentration range from 0.73 μM to 14.7 μM for emetine and 4.41 μM to 8.82 μM for pH-type liposomes with fluorescent emetine per emetine. Photographs of fish from each group were taken at the final time point to monitor the intensity of fluorescence from absorbed emetine. A Discovery V8 stereoscopic optical microscope and Zeiss equipment were used for the observations. The structure of the fluorescently labelled emetine used in the experiment is shown in FIG. 3.1. The results are shown in FIG. 3.2.

The amount of absorbed emetine increases with dose (1.47, 3.67, 7.35 μM (1, 2.5, 5 μg/ml)). It is observed within the yolk of the larvae, especially in the liver and intestine region. However, when emetine was in pH liposomes, uptake increased significantly. In addition to the yolk, some amounts were detected in parts of the fish head. The concentration of emetine encapsulated in liposomes was 4.41 and 8.82 μM.

As a result of the observations made, it should be concluded that pH liposomes improve the safety of emetine and its absorption.

### EXAMPLE IX

DPPC/Cholesterol/DSPE-PEG-NH2 (15 pH MX) Liposomes—Loading with Mitoxantrone.

A solution of lipids in ethanol was prepared by weighing 106 mg of DPPC, 27.9 mg of cholesterol and 16.1 mg of DSPE-PEG(2000)NH2 and dissolving in 1.5 ml of ethanol. The total was then heated for 15 minutes at 60° C.

0.69 g (NH)₄₂ SO₄, was weighed in sterile urea and dissolved in 15 ml of deionised water. The pH of the solution was 5.5. The solution was filtered into sterile urea using sterile syringe filters with a hydrophilic PTFE membrane with a pore diameter of 0.22 μm.

Synthesis was carried out in a NanoAssemblr IGNITE microfluidic Device™ with an NxGen cartridge setting the following flow conditions: aqueous to organic phase volume ratio 9:1; total flow rate 5 ml/min, final volume of reaction mixture 9.1 ml; initial loss 0.85 ml; final loss 0.05 ml. The hydrodynamic diameter and PDI of the resulting liposomes were then measured.

Liposomes were then dialysed in PBS buffer pH 7.4. A 5-fold PBS buffer concentrate pH 7.4 was prepared by dissolving 20 g NaCl, 3.6 g Na₂ HPO₄ (anhydrous), 0.775 g NaH₂ PO×2H₄₂ O (dihydrate), 0.5 g KCl in 0.5 L deionised water (milliQ). Mixed on a magnetic stirrer for 30 min. The finished concentrate was diluted five times. The pH of the buffer was 7.4. Dialysis membranes with a pore diameter of MWCO 100 kD and a volume of 10 ml each were conditioned for 20 min each time in solutions, successively: 10% ethanol, deionised water and PBS buffer, pH 7.4. The conditioning solutions were previously filtered through sterile syringe filters with a hydrophilic PTFE membrane with a pore diameter of 0.22 μm. Liposomes were dialysed for 16 h at 4° C. After 3 h, the buffer was replaced and dialysis continued for 13 h.

Subsequently, a starting solution of mitoxantrone dihydrochloride was prepared at a concentration of 50 mg/ml. Then 20 uL of emetine hydrochloride stock solution was added to the liposomes for each 1 ml of liposomes and mixed. The liposomes were heated in a 55° C. water bath for 15 minutes. After removal from the water bath, the liposomes were allowed to cool for 30 minutes. The vessel with liposomes was protected from light. Liposomes were filtered through sterile syringe filters with a hydrophobic PTFE membrane with a pore diameter of 0.45 μm. 0.5 ml of each sample was separated on a Sephadex G-50 packed chromatography column and % EE drug encapsulation rates were calculated by determining the free drug and drug bound in the liposomes using UV-Vis spectrophotometry against a calibration curve. The % EE ratio was 99.9%. The rest of the liposomes were dialysed in PBS buffer pH 7.4 for 16 h changing the dialysis solution three times after 1 h, 3 h and 15 h. The hydrodynamic radius, polydispersion coefficient and Zeta potential of the liposomes at each stage of synthesis were investigated using dynamic light scattering. The results are summarised in Table 7.1 and FIGS. 3.1.-3.4.

Table 7.1. Physical Parameters (Hydrodynamic Diameter, Polydispersion Coefficient and Zeta Potential) of 15 pH MX Liposomes after Successive Synthesis Steps.

TABLE 7.1
Physical parameters (hydrodynamic diameter, polydispersion
coefficient and Zeta potential) of 15 pH MX liposomes
after successive synthesis steps.

| | Hydrodynamic | | Zeta |
|---|---|---|---|
| | diameter | | potential |
| Synthesis stage | [nm] | PDI | [mV] |
| Stage I - Microfluid | 86.79 ± 1.41 | 0.045 ± 0.009 | 9.70 ± 0.26 |
| synthesis | | | |
| Step II - change of | 83.61 ± 2.3 | 0.116 ± 0.044 | 6.56 ± 0.49 |
| buffer to PBS pH 7.4 | | | |
| Stage III - drug loading | 83.82 ± 1.59 | 0.018 ± 0.02 | 5.04 ± 0.23 |

No significant changes in hydrodynamic diameters and PDI coefficients were observed, indicating the stability of the liposomes during synthesis and the absence of carrier aggregation. The similar Zeta potential value of the liposomes before and after loading with positively charged mitoxantrone dihydrochloride is indicative of drug loading into the centre of the liposomes and not drug deposition in the outer polymer layer of the carrier.

Subsequently, the leakage rate of mitoxantrone from 15 pH MX liposomes in the presence of albumin was investigated:
bovine BSA—albumin used in vitro as an additive to cell medium
rat RSA—the albumin available in rat blood during in vivo studies.
Human HSA—a protein found in approximately 5% of human blood

The results are summarised in Table 7.2.

% of released EM from liposomes
(24 h incubation. 37° C., 5% protein)
[%]

| Sample code | BSA | RSA | HSA |
|---|---|---|---|
| 15 pH MX | 17.3 | 12.3 | 17.1 |

The 15 pH MX carrier showed high stability in the presence of bovine serum albumin (BSA) rat serum albumin (RSA) and human serum albumin (HSA). No sudden leakage of drug from the carrier was observed and the drug release rate was classified as less than 17.3%, indicating that these liposomes can be used as carriers for extended-release mitoxantrone.

### EXAMPLE X

### In Vivo Tests for 15 pH MX Liposomes

Female mice of the NOD.Cg-Prkdc strain^{scid} II2rgtm1Sug/JicTac strain were injected with tumour cells of the CAL-51 line (2×10⁶/mouse) after intratumoural engraftment when the tumour volume reached approximately 56-60 mm³, the administration of 15 pH MX nanoparticles or free mitoxantrone at a concentration identical to that of the drug in 15 pH liposomes was initiated. Nanoparticles were administered intravenously every 2 weeks, for a total of 4 times. Administration of free mitoxantrone was started on the same day as the liposomes, but was administered at twice the frequency (1× per week), for a total of 7 times. The fixed dose of mitoxantrone was 0.625 mg/kg. Tumour volume measurements were then taken over time.

Tumour volume was measured percutaneously with an electronic calliper in the shorter and longer dimension and converted to volume according to the formula:

$$Tumour volume = {a}^{2} \times b / 2$$

where: a and b are the shorter and longer dimensions in mm, respectively. The measurement groups consisted of eight individuals each. The mean tumour volume was extracted from each group.

The results are shown in FIG. 4.1.

The 15 pH-MX liposomes markedly slow tumour growth. The average tumour volume is three times lower than the average tumour volume of untreated mice by 1.5 times lower than the tumour volume of mice treated with free mitoxantrone.

### EXAMPLE XI

### Emetine Loading of DOPC/DPPC/Cholesterol/DSPE-PEG-NH2 Liposomes

34.1 mg DPPC, 12.8 mg cholesterol, 7.4 mg DSPE-PEG(2000)NH2, 15.7 mg DOPC were weighed into glass bottles and dissolved in 1 ml ethanol. The whole mixture was heated for 15 minutes in a water bath at 60° C.

Preparation of a 250 mM aqueous solution (NH)₄₂ SO₄: 0.661 g (NH)₄₂ SO₄, was weighed in sterile urea and dissolved in 20 ml of deionised water. The solution was filtered into the sterile urea using sterile syringe filters with a hydrophilic PTFE membrane with a pore diameter of 0.22 μm. Part of the filtered solution was poured into a phalcon and the pH was measured, which was 5.3.

9 ml of ammonium sulphate solution was taken into a 10 ml syringe. A hot lipid solution was drawn into a 1 ml syringe. Both syringes were placed in a NanoAssemblr IGNITE microfluidic Device™ with an NxGen cartridge and the mixing of the solutions began immediately. Flow parameters set: volume ratio of aqueous phase to organic phase 9:1; total flow rate 5 ml/min, final volume of reaction mixture 9.05 ml; initial loss 0.9 ml; final loss 0.05 ml. After synthesis, the liposomes were dialysed in phosphate buffer pH 8.5 for 12 h changing the external buffer twice. Buffer preparation: a 5-fold PBS buffer concentrate pH 7.4 was prepared by dissolving 20 g NaCl, 3.6 g Na₂ HPO₄ (anhydrous), 0.775 g NaH₂ PO×2H₄₂ O (dihydrate), 0.5 g KCl in 0.5 L deionised water (milliQ). The mixture was stirred on a magnetic stirrer for 30 min. The concentrate was then diluted fivefold and 0.1 M NaOH was added until a pH of 8.5 was obtained. MWCO 100 kD membranes in a volume of 10 ml were used for dialysis and conditioned for 20 min each time in solutions of, successively: 10% ethanol, deionised water and PBS buffer, pH 8.5. The conditioning solutions were previously filtered through sterile syringe filters with a hydrophilic PTFE membrane with a pore diameter of 0.22 μm. Subsequently, 25 uL of emetine solution with DMSO at a concentration of 50 mg/ml was added to the liposomes for every one millilitre of liposomes after dialysis. The liposomes were left without light in the refrigerator at approximately 4° C. for 24 h. Subsequently, the liposomes were purified on Sephadex G-25 packed chromatography columns and the encapsulation rate % EE=71% was determined. Size and PDI were measured using dynamic light scattering. This is shown in FIG. 5.1.

The addition of unsaturated lipids to the lipid core structure does not affect the physical parameters of the liposomes (size and PDI factor) and has little effect on the loading of emetine into the liposomes, with % EE of emetine in the liposomes more than 20% higher than literature values.

### EXAMPLE XII

DOPG/DPPC/Cholesterol/DSPE-PEG-NH2 Liposomes Loaded with Emetine and Mitoxantrone

The effect of unsaturated and negatively charged DOPG-type lipids on % EE values of emetine and mitoxantrone was investigated.

34. mg DPPC, 12.8 mg cholesterol, 7.4 mg DSPE-PEG(2000)NH2, 15.8 mg DOPG were weighed into glass bottles and dissolved in 1 ml ethanol. The whole mixture was heated for 15 minutes in a water bath at 60° C.

An aqueous solution (NH)₄₂ SO₄ with a concentration of 350 mM was prepared. The solution was filtered into sterile urea using sterile syringe filters with a hydrophilic PTFE membrane with a pore diameter of 0.22 μm. Part of the filtered solution was poured into a phalcon and the pH was measured. The pH was 5.3.

Performing liposome synthesis: 9 ml of ammonium sulphate solution was taken into a 10 ml syringe. A hot lipid solution was drawn into a 1 ml syringe. Both syringes were placed in a NanoAssemblr IGNITE microfluidic Device™ with an NxGen cartridge and the mixing of the solutions was started immediately. Flow parameters set: volume ratio of aqueous phase to organic phase 9:1; total flow rate 5 ml/min, final volume of reaction mixture 9.05 ml; initial loss 0.9 ml; final loss 0.05 ml. After synthesis, the liposomes were dialysed in phosphate buffer pH 8.5 for 12 h changing the external buffer twice. Buffer preparation: a 5-fold PBS buffer concentrate pH 7.4 was prepared by dissolving 20 g NaCl, 3.6 g Na₂ HPO₄ (anhydrous), 0.775 g NaH₂ PO×2H₄₂ O (dihydrate), 0.5 g KCl in 0.5 L deionised water (milliQ). The mixture was stirred on a magnetic stirrer for 30 min. The concentrate was then diluted fivefold and 0.1 M NaOH was added until a pH of 8.5 was obtained. MWCO 100 kD membranes in a volume of 10 ml were used for dialysis and conditioned for 20 min each time in solutions of, successively: 10% ethanol, deionised water and PBS buffer, pH 8.5. The conditioning solutions were previously filtered through sterile syringe filters with a hydrophilic PTFE membrane with a pore diameter of 0.22 μm. Subsequently, 25 uL of emetine solution in DMSO with a concentration of 50 mg/ml was added to the liposomes for every one millilitre of liposomes after dialysis and then 12.5 uL of mitoxantrone solution in DMSO with a concentration of 50 mg/ml also for every ml of liposome solution after dialysis. Liposomes were heated in a water bath at 60° C. for 30 minutes. Subsequently, the liposomes were purified on Sephadex G-25 packed chromatography columns and the % EE encapsulation coefficient was determined by UV-Vis spectroscopy against the calibration curves using the principle of additivity of absorbance. The determined % EE ratios were: 82.3% for emetine and 98.9% for mitoxantrone. The size and PDI ratio were measured using dynamic light scattering. This is shown in FIG. 6.1.

The presence of negatively charged lipids does not affect the high loading of emetine and mitoxantrone into the core of liposomes.

### EXAMPLE XIII

M7-M10(pH) and M2-M5(HSA) Liposomes Loaded with Emetine and Mitoxantrone

### Stage I.

Preparation of the lipid solution: 70.6 mg of DPPC, 18.6 mg of cholesterol, 10.7 mg of DSPE-PEG(2000)NH2 were weighed out in glass bottles and dissolved in 1 ml of ethanol; the whole was heated for 15 minutes in a water bath at 60° C.

Preparation of the aqueous solution: 0.925 g of (NH)₄₂ SO₄, was weighed in sterile urea and dissolved in 20 ml of deionised water; the solution was filtered into sterile urea using sterile syringe filters with a hydrophilic PTFE membrane with a pore diameter of 0.22 μm, then part of the filtered solution was transferred to a flask and the pH was measured.

Performing liposome synthesis: 9 ml of ammonium sulphate solution was drawn into a 10 ml syringe and a hot lipid solution was drawn into a 1 ml syringe, after which both syringes were placed in a NanoAssemblr IGNITE microfluidic Device™ with an NxGen cartridge and the mixing of the solutions was immediately started with the set parameters: volumetric ratio of aqueous phase to organic phase 9:1; total flow rate 5 ml/min, final volume of reaction mixture 9.05 ml; initial loss 0.9 ml; final loss 0.05 ml.

For further studies, 0.15 ml of liposomes were left after synthesis. The remaining liposomes were used in step II.

### Stage II.

Liposome dialysis after synthesis (microfluidics)—external buffer exchange A 5-fold PBS buffer concentrate pH 7.4 was prepared by dissolving 20 g NaCl, 3.6 g Na₂ HPO₄ (anhydrous), 0.775 g NaH₂ PO×2H₄₂ O (dihydrate), 0.5 g KCl in 0.5 L deionised water (milliQ). The mixture was stirred on a magnetic stirrer for 30 min. Then, 400 ml of PBS buffer concentrate (5×) was measured using a measuring cylinder and transferred to a beaker. Subsequently, 1600 ml of distilled water was added. 0.1 M NaOH was added to the buffer until a pH of 7.4 was obtained with an acceptable pH range of 7.38-7.42. MWCO 100 kD pore diameter dialysis tubular membranes of 10 ml volume were conditioned for 20 minutes each time in solutions, successively: 10% ethanol, deionised water and PBS buffer, pH 7.4. The conditioning solutions were previously filtered through sterile syringe filters with a hydrophilic PTFE membrane with a pore diameter of 0.22 μm.

The synthesised liposomes were then placed in the membrane. A mixing element and the membrane with the liposomes were placed in a glass bottle containing PBS pH 7.4. The volume of PBS buffer was chosen so that the buffer completely surrounded the liposomes in the membrane. The bottle was placed on a magnetic stirrer in a refrigerator (4° C.). After 3 hours, the buffer was replaced and dialysis continued for 12 hours. 0.15 ml of liposomes were left after dialysis for further studies. The remaining liposomes were used in step Ill. In order to reuse the membrane in the further synthesis step, the dialysis membrane was conditioned in PBS pH 7.4 (buffer was exchanged twice).

Stage III. Encapsulation of Drugs into Liposomes.

Stock solutions of emetine hydrochloride at 50 mg/ml and mitoxantrone dihydrochloride in DMSO were prepared. Then 42.5 μl of emetine hydrochloride stock solution was added to 8.5 ml of liposomes after dialysis in PBS, pH 7.4, and mixed. Then 170 μl of mitoxantrone dihydrochloride stock solution was added and mixed. The bottle was tightly closed and heated in a water bath at 60° C. for 15 minutes. After removal from the water bath, the liposomes were allowed to cool for 30 minutes. The vessel with the liposomes was protected from light.

The liposomes were then filtered through a sterile syringe filter with a 0.45 μm PTFE membrane (hydrophobic) into a sterile phalcon. The liposomes were divided into two solutions of 3 ml each: Liposomes M2-M5 and Liposomes M7-M10. A solution of 10% HSA in water was added to Liposomes M2-M5 at a volume ratio of liposomes to HSA solution of 3:1. Liposomes M1-M5 were then incubated for 1 h at 25° C. Drug encapsulation rates into liposomes were determined by separating 0.2 mL of sample M2-M5 and M7-M10 on Sephadex G-25 packed chromatography columns. Initially, 8 fractions of 1 mL each were collected by eluting with PBS buffer pH 7.4. Fractions containing liposomes were detected by UV-Vis spectroscopy and then pooled together and concentrations were determined by spectroscopic and HPLC methods. The rest of the fractions were pooled together and the free drug was further eluted from the column to a volume of 25 ml. The concentration of the free drug was determined by UV-Vis spectroscopy against a calibration curve using the principle of absorbance additivity. As a quality control measure, the drug concentration of the samples without separation on the column was also determined using HPLC.

Subsequently, both batches of liposomes were dialysed in PBS buffer pH 7.4 for 16 h exchanging dialysis solutions three times after 1 h, 3 h and 15 h. Membrane solutions were filtered through a sterile syringe filter with a hydrophilic PTFE membrane with a pore diameter of 0.22 μm sterile phalcones. The drug concentration in the liposomes was again determined by HPLC. The hydrodynamic radius, polydispersion coefficient and zeta potential of the liposomes at each synthesis step were investigated using dynamic light scattering. The results are summarised in Table 8.1, Table 8.2 and FIGS. 7.1-7.8.

TABLE 8.1
Change in physical parameters of M2-M5 and M7-M10
liposomes during the synthesis steps.

| | Hydrodynamic | | Zeta |
|---|---|---|---|
| | diameter | | potential |
| The synthesis stage | [nm] | PDI | [mV] |
| Stage I - Microfluid | 80.49 ± 0.67 | 0.027 ± 0.023 | 8.92 ± 0.55 |
| synthesis | | | |
| Step II - change of buffer | 85.53 ± 0.17 | 0.003 ± 0.001 | 6.07 ± 0.22 |
| to PBS pH 7.4 | | | |
| Stage III - Drug loading - | 85.27 ± 0.58 | 0.020 ± 0.016 | 5.51 ± 0.25 |
| M7-M10 liposomes | | | |
| Stage II - drug loading - | 86.07 ± 0.74 | 0.030 ± 0.018 | 5.75 ± 0.55 |
| M2-M5 liposomes | | | |

TABLE 8.2
Quantities of emetine hydrochloride (EM) and mitoxantrone
dihydrochloride (MX) added to the synthesis, together
with drug encapsulation factors and the final
ratio of EM to MX concentrations.

| | EM | MX | | | |
|---|---|---|---|---|---|
| Sample | additive | additive | % EE EM | % EE MX | |
| code | [mg]. | [mg]. | [%] | [%] | CEM/CMX |
| M2-M5 | 0.24 | 0.9 | 90 | 97 | 4.04 |
| M7-M10 | 0.24 | 0.9 | 98 | 100 | 3.83 |

Liposomes are characterised by a very high encapsulation ratio for both drugs with a weight ratio of mitoxantrone to emetine of 3.75:1. The absence of changes in the values of hydrodynamic diameters and PDI polydispersity coefficients testifies to the stability of the nanoparticles during all stages of synthesis. The lack of change in Zeta potential values before and after loading of the drug into the nanoparticles confirms the loading of the drug into the interior of the liposomes and not the interaction with the outer shell of the liposomes. The slight difference in the Zeta potential values of HSA-coated and protein-free liposomes may indicate protein incorporation into the outer layer of the polymer envelope without protein attachment to the free amino groups of the DSPC-PEG(2000)NH2 derivative. This also suggests no significant change in the hydrodynamic diameter of the liposomes after the HSA-coating process.

The stability of formulations M2-M5 and M7-M10 was investigated at: 1 week, 2 weeks, 3 weeks, 4 weeks and 2, 3, 4, 5 and 6 months during refrigerated storage at 2-8° C.

The following parameters were checked at the selected time point:
size (HR hydrodynamic diameter) together with the difference between the selected time point and point 0 and the polydispersity index (PDI)—the results are shown in Table 8.3,
total concentration of emetine and mitoxantrone by LC-MS, HPLC methods—results are shown in Table 8.4,
Drug leakage: drug concentration inside liposomes and outside liposomes by LC-MS, HPLC methods—results are shown in Table 8.5.

| | M2-M5 | | | | | M7-M10 | | | | |
|---|---|---|---|---|---|---|---|---|---|---|
| | HR | ±HR | | | change in | HR | ±HR | | | change in |
| Time | [nm]. | [nm]. | PDI | ±PDI | HR [%] | [nm]. | [nm]. | PDI | ±PDI | HR [%] |
| 0 | 86.07 | 0.74 | 0.03 | 0.018 | 0 | 85.27 | 0.58 | 0.02 | 0.016 | 0.0 |
| 7 days | 86.8 | 0.71 | 0.056 | 0.012 | 0.8 | 86.81 | 1.41 | 0.017 | 0.005 | 1.8 |
| 14 days | 89 | 1.99 | 0.052 | 0.011 | 3.4 | 87.27 | 2.08 | 0.004 | 0.001 | 2.3 |
| 21 days | 87.8 | 1.3 | 0.024 | 0.005 | 2.0 | 87.72 | 0.48 | 0.008 | 0.002 | 2.9 |
| 1 month | 86.35 | 1.26 | 0.009 | 0.007 | 0.3 | 84.17 | 1.28 | 0.047 | 0.023 | −1.3 |
| 2 months | 85.78 | 0.93 | 0.047 | 0.022 | −0.3 | 86.17 | 0.63 | 0.023 | 0.015 | 1.1 |
| 3 months | 86.08 | 1.37 | 0.081 | 0.007 | 0.0 | 89.01 | 1.36 | 0.038 | 0.014 | 4.4 |
| 4 months | 83 | 1.22 | 0.084 | 0.016 | −3.6 | 84.38 | 1.19 | 0.029 | 0.009 | −1.0 |
| 5 months | 81.75 | 0.99 | 0.111 | 0.005 | −5.0 | 84.72 | 0.406 | 0.034 | 0.004 | −0.6 |
| 6 months | 87.83 | 1.80 | 0.085 | 0.018 | 2.0 | 88.51 | 2.17 | 0.057 | 0.008 | 3.8 |

| | M2-M5 | | | | M7-M10 | | | |
|---|---|---|---|---|---|---|---|---|
| | Ctotal_EM | Change | Ctotal_MTX | Change | Ctotal_EM | Change | Ctotal_MTX | Change |
| Time | [μg/ml]. | [%] | [μg/ml]. | [%] | [μg/ml]. | [%] | [μg/ml]. | [%] |
| 0 | 164.3 | 0.0 | 568.62 | 0.0 | 193.1 | 0.0 | 882.00 | 0.0 |
| 7 days | 156.5 | −4.7 | 563.5 | −0.9 | 214.4 | 11.0 | 921.6 | 4.5 |
| 14 days | 160.20 | −2.5 | 565.20 | −0.6 | 212.10 | 9.8 | 904.40 | 2.5 |
| 21 days | 174.80 | 6.4 | 549.10 | −3.4 | 242.30 | 25.5 | 941.30 | 6.7 |
| 1 month | 159.40 | +3.0 | 549.20 | −3.4 | 240.00 | 24.3 | 945.20 | 7.2 |
| 2 months | 162.80 | −0.9 | 558.90 | −1.7 | 186.20 | −3.6 | 881.30 | −0.1 |
| 3 months | 159.30 | −3.0 | 543.80 | −4.4 | 240.20 | 24.4 | 860.30 | −2.5 |
| 4 months | 157.03 | −4.4 | 578.62 | 1.8 | 197.96 | 2.5 | 844.06 | −4.3 |
| 5 months | 165.09 | 0.5 | 552.33 | −2.9 | 230.30 | 19.3 | 828.97 | −6.0 |
| 6 months | 159.31 | −3.0 | 584.01 | 2.7 | 218.15 | 13.0 | 863.81 | −2.1 |

TABLE 8.5
Change in percentages of emetine hydrochloride and mitoxantrone
dihydrochloride remaining in the free drug fraction after separation
of M2-M5 and M7_M10 liposomes on SPE columns during
storage of liposomes at 2-8° C.

| | |
|---|---|
| M2-M5 | M7-M10 |

| Time | EM [%] | MTX [%] | EM [%] | MTX [%] |
|---|---|---|---|---|
| 0 | 24.8 | 29.2 | 24.5 | 35.4 |

| | | | | | |
|---|---|---|---|---|---|
| 7 | days | 23.1 | 28.2 | 26.4 | 34.7 |
| 14 | days | 23.7 | 25.4 | 30.5 | 32.2 |
| 21 | days | 33.5 | 36.1 | 27.3 | 28.1 |
| 1 | month | 31.0 | 31.7 | 36.1 | 35.6 |
| 2 | months | 28 | 28.5 | 27 | 27.9 |
| 3 | months | 19.6 | 19.8 | 24.9 | 25.1 |
| 4 | months | 15.90 | 13.24 | 22.83 | 19.21 |
| 5 | months | 15.9 | 18.5 | 21.4 | 32.2 |
| 6 | months | 14.65 | 15.87 | 19.59 | 20.4 |

Liposomes M2-M5 and M7_M10 do not show: aggregation characteristics, drug leakage or drug degradation in liposomes and are stable in storage at 2-8° C. over 6 months.

In vivo tests were performed for Liposomes M2-M5 and M7-M10.

Female mice of the NOD.Cg-Prkdc strain^{scid} II2rgtm1Sug/XicTac were injected with tumour cells of the CAL-5I line (2×10⁶/mouse) after dosimal implantation of tumour cells when tumour volume reached approximately 56-60 mm³, the administration of M2-M5 and M7-M10 nanoparticles or free drugs at concentrations identical to the drug concentrations in M2-M5 and M7-M10 liposomes was initiated. Nanoparticles were administered intravenously every 2 weeks, for a total of 4 times. Free drug administration started on the same day as the liposomes but was administered at twice the frequency (1× per week), for a total of 7 times. Fixed dose of mitoxantrone: 0.625 mg/kg body weight and emetine 0.179 mg/kg body weight. Tumour volume measurements were then taken over time.

Tumour volume was measured percutaneously with an electronic calliper in the shorter and longer dimension and converted to volume according to the formula:

$$Tumour volume = {a}^{2} \times b / 2$$

where: a and b are the shorter and longer dimensions in mm, respectively. The measurement groups consisted of eight individuals each. The mean tumour volume was extracted from each group.

The results are shown in FIG. 7.9. Both M7_M10 and M2-M5 liposomes clearly slow down the increase in tumour volume. This effect is particularly evident for the M7_M10 nanoparticles where the average tumour volume is four times smaller than the average tumour volume in mice untreated or treated with a mixture of mitoxantrone and emetine not encapsulated in a liposomal nanoparticle.

### EXAMPLE XIV

Synthesis of Staurosporine-Loaded BST Liposomes BEMs Loaded with Emetine and BSTEMs Loaded with Emetine and Staurosporine.

### Stage I.

A lipid solution was prepared in 99.8% ethanol at 100 mg/ml with the composition: DPPC/Cholesterol/DSPE-PEG(2000)NH2 in molar fractions of 0.65/0.32/0.026 and mass fractions of 0.70650/0.18607/0.10743. The whole was heated for 15 minutes in a water bath at 60° C. 2.313 g (NH)₄₂ SO₄, was weighed in sterile urea and dissolved in 50 ml of deionised water. The solution was filtered using sterile syringe filters with a hydrophilic PTFE membrane with a pore diameter of 0.22 μm. The PH of the solution was titrated with 0.3M HCL solution until a pH of 5.3 was obtained.

Performing liposome synthesis: the synthesis was carried out in a NanoAssemblr Blaze™ microfluidic device with a High Flow NxGen cartridge at the set flow parameters: aqueous to organic phase volume ratio 5:1, total flow rate 103 ml/min, final volume of the reaction mixture 40 ml including an initial loss of 1 ml and a final loss of 1 ml. The hydrodynamic radius of the resulting liposomes and the polydispersion coefficient were measured using dynamic light scattering. The results are shown in FIG. 8.1. Dialysis buffers were prepared:
Acetate buffer: 9.575 g CH₃ COONa×3H₂O was weighed and dissolved in 998.13 ml deionised water, then 1.865 ml glacial acetic acid was added,
phosphate buffer: 5-fold PBS buffer concentrate pH 7.4 was prepared by dissolving 20 g NaCl, 3.6 g Na₂ HPO₄ (anhydrous), 0.775 g NaH₂ PO×2H₄₂O (dihydrate), 0.5 g KCl in 0.5 L deionised water (milliQ) and stirred on a magnetic stirrer for 30 min, then 400 ml of PBS buffer concentrate (5×) was measured using a measuring cylinder and transferred to a beaker; successively, 1600 ml of distilled water was added; 0.1 M NaOH was added to the buffer until a pH of 7.4 was obtained with an acceptable pH range of 7.38-7.42.

Dialysis tubular membranes with a pore diameter of MWCO 100 kD and a volume of 10 ml were conditioned for 20 minutes each time in solutions of, respectively: 10% ethanol, deionised water and acetate (BST and BSTEM) or phosphate (BEM) buffer. The conditioning solutions were previously filtered through sterile syringe filters with a hydrophilic PTFE membrane with a pore diameter of 0.22 μm.

### BST and BSTEM Synthesis

Then 10 ml of synthesised liposomes were placed in a membrane conditioned in acetate buffer. The liposomes were dialysed in acetate buffer for 2 hours at 4° C. to 7° C., changing the external buffer once after one hour. Subsequently, 50 uL of DMSO and 20 uL of Staurosporine solution in DMSO at a concentration of 40 mg/ml per 1 mL of liposomes were added to the liposomes. Staurosporine solution was added in portions of 20 uL, stirring each time until the precipitate disappeared. The liposomes were then left for 1 h at room temperature. The size and polydispersity coefficient of the liposomes were controlled by dynamic light scattering. An example curve is provided in FIG. 8.2.

Subsequently, the sample was divided into two parts. The first part (BST liposomes) was purified by dialysis in PBS buffer pH 7.4 for 12 h by changing the external solution twice after 2 h and 6 h. Staurosporine concentration in the liposomes was then determined by UV-Vis spectroscopy against a calibration curve, by dissolving 50 uL of the liposome sample in 900 uL8 ethanol with 50 uL of 0.3M HCl and the spectrum was measured. The liposomes were named BST. From the second part, excess Staurosporine was purified by dialysis in PBS buffer pH 8.5 for 12 h at 4° C. changing the external buffer twice after 2 h and 6 h. Elution of unbound Staurosporine occurred simultaneously with the change of external buffer. From the purified Staurosporine-loaded liposomes, a 50 ul sample was taken to which 50 ul of 0.3 M HCl solution and 900 ul of ethanol were added and the samples were then diluted 10-fold. Staurosporine concentration in liposomes was determined by UV-Vis spectroscopy against a calibration curve at 291 nm. The spectrum of the liposomes is shown in FIG. 8.5 (black curve). The concentration of Staurospotin in the liposomes was 1.37 mM. To the rest of the liposomes, 20 uL of emetine hydrochloride solution at a concentration of 50 mg/ml in water was added for each 1 ml of liposomes, mixed and the whole was placed in the refrigerator for 16 h. Subsequently, the liposomes were purified on a chromatography column and the % EE encapsulation factor of emetine and staurosporine was determined by UV-Vis spectroscopy against a calibration curve. The final liposomes were filtered through sterile 0.22 um syringe filters with a hydrophilic PTFE membrane.

The drug concentration of the liposomes was determined by UV-Vis spectroscopy using the principle of additivity of absorbance and numerical methods by dissolving 50 ul of the liposome sample in 900 ethanol with the addition of 50 ul of 0.3M HCl solution and then diluting the samples 10 times. The hydrodynamic diameter, polydispersion coefficient and zeta potential of the final liposomes were measured. The data of the final BSTEM liposomes are shown in FIGS. 8.3 and 8.4. Table 8.7 shows the changes in hydrodynamic diameters and polydispersity coefficients after the different synthesis steps.

TABLE 8.7
Changes in hydrodynamic diameters and polydispersity
coefficients after the different steps in the
synthesis of BST and BSTEM liposomes

| | | Hydrodynamic | | | |
|---|---|---|---|---|---|
| | | diameter | | | |
| L.P | Synthesis stage | [nm] | SD | PDI | SD |
| 1 | Synthesis - | 105.5 | 1.09 | 0.095 | 0.024 |
| | microfluidics | | | | |
| 2 | Dialysis in acetate | 121.7 | 1.10 | 0.092 | 0.005 |
| | buffer pH 5 | | | | |
| 3 | After the addition | 130.5 | 0.19 | 0.094 | 0.024 |
| | of DMSO and | | | | |
| | Staurosporine | | | | |
| 4 | Dialysis in PBS | 110.0 | 0.78 | 0.113 | 0.011 |
| | buffer pH 7.4 | | | | |
| | (BST liposomes) | | | | |
| 5 | Dialysis in PBS | 109.0 | 0.87 | 0.189 | 0.066 |
| | buffer pH 8.5 + | | | | |
| | addition of | | | | |
| | Emetine | | | | |
| 6 | Dialysis in PBS | 104.9 | 1.12 | 0.109 | 0.024 |
| | buffer pH 7.4 - | | | | |
| | final purification | | | | |
| | (BSTEM liposomes) | | | | |

### BEM Synthesis

Another 10 ml of synthesised liposomes were placed in a separate membrane conditioned in phosphate buffer. The liposomes were dialysed in phosphate buffer for 2 hours (temperature 4° C. to 7° C.) changing the external buffer once after one hour. Then 20 ul of emetine hydrochloride solution in water (50 mg/ml) was added to the liposomes and the whole was left for 24 h in the refrigerator. The liposomes were separated on chromatography columns using phosphate buffer pH 7.4 as eluent and the % EE encapsulation factor was determined against the calibration curve. The final liposomes were filtered through sterile 0.22 um syringe filters with a hydrophilic PTFE membrane. The concentration of emetine hydrochloride was determined by UV-Vis spectroscopy against the calibration curve by diluting the sample under identical conditions to the BST and BSTEM samples. Hydrodynamic diameters and PDI coefficient were measured. The drug concentrations in the final samples were respectively: 1.22 mM staurosporine in BST nanoparticles, 1.1 mM staurosporine and 1.24 mM emetine in BSTEM nanoparticles and 1.26 mM emetine in BEM nanoparticles.

### EXAMPLE XV

Comparison of Toxicity of Emetine or Staurosporine Alone with Emetine or Staurosporine in BpH Liposomes (BEM and BST)—In Vivo Studies

In order to compare the toxicity of emetine and staurosporine with the same drugs loaded into BpH liposomes, an acute toxicity test was conducted on Zebrafish (Danio rerio). The test was designed to determine the acute or lethal toxicity of the chemicals on the embryonic stages of the fish. Briefly, zebrafish larvae were treated with emetine and BES liposomes or staurospoine and staurosporine with BST liposomes for a period of 96 hours. A static system was used in the experiment, as changes in solution concentrations did not exceed a range of 20% of the nominal concentrations.

The embryo culture fluid and solution preparation was embryo culture medium (E3 solution: 5 mmol/L NaCl, 0.17 mmol/L KCl, 0.33 mmol/L CaCl₂ and 0.33 mmol/L MgSO₄, containing no methylene blue, and with a pH value of approximately 7.2). The experiment was carried out in 24-well plates, 5 embryos per well, 10 per group. The plate was covered and kept in an incubator set at 28±0.5° C. with a light/dark period of 12/12 hours. During the exposure period, larvae were tracked every 24 h and four visual observations, including coagulation of fertilised eggs, lack of somite formation, lack of separation of tail bud from yolk sac and lack of heartbeat, were recorded as indicators of mortality. At the end of the exposure period (96 hpf), acute toxicity was determined based on a positive result from any of the four recorded visual observations and the LC50 was calculated. Liposomes alone without drug loading were also tested.

Figure FIG. 9.1 shows a comparison of toxicity after 96 hours of exposure to emetine and BEM liposomes with emetine. The half maximum lethal concentration (LC50) calculated for emetine was 19 μl/ml, while for emetine encapsulated in BEM liposomes it was 17 μl/ml.

Figure FIG. 9.2 shows a comparison of the toxicity of staurosporine and liposomes with staurosporine BST after 96 hours of fish incubation. The LC50 value for staurosporine alone was 17 nmol/L while for staurosporine in liposome the value was 24 nmol/L

The conclusion to be drawn from the comparison is that liposomes improve the safety of emetine and staurosporine and are not themselves toxic.

1. A modified liposome for drug delivery characterised in that it is composed of three major lipids, namely DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine (ammonium salt of 1,2-distearoyl-sn-glycero-3-phosphoethanolamine-N-[amino(polyethylene glycol)-2000]) in an amount of 10.6 to 10.7% by weight and at least one lipid from the group consisting of DOPC (dioleoylphosphatidylglycerol) and DOPG (dioleoylphosphatidylcholine), with a DOPC (dioleoylphosphatidylglycerol) content in the liposome of not more than 22.4% by weight and a DOPG (dioleoylphosphatidylcholine) content in the liposome of not more than 22.6% by weight.

2. The liposome according to claim 1, characterised in that the DOPC (dioleoylphosphatidylglycerol) content of the liposome ranges from 7.5 to 22.4 wt. %.

3. The liposome according to claim 1, characterised in that the content of DOPG (dioleoylphosphatidylcholine) in the liposome ranges from 7.5 to 22.6 wt. %.

4. The liposome according to claim 1, characterised in that its outer surface is coated with a layer of human serum albumin (HSA).

5. A liposome composed of at least three lipids, including DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine (ammonium salt of 1,2-distearoyl-sn-glycero-3-phosphoethanolamine-N-[amino(polyethylene glycol)-2000]) in an amount of 10.6 to 10.7% by weight for use in the transfer of at least one drug, in particular one with anticancer activity.

6. The use according to claim 5, characterised in that the liposome contains DOPC in an amount of not more than 22.4 wt. %.

7. The use according to claim 5, characterised in that the liposome contains DOPG (dioleoylphosphatidylcholine) in an amount of not more than 22.6% by weight.

8. The use according to claim 5, characterised in that the outer surface of the liposome is coated with a layer of human serum albumin (HSA).

9. The use according to claim 5, characterised in that the liposome is used to carry emetine.

10. The use according to claim 5, characterised in that the liposome is used to carry mitoxantrone.

11. The use according to claim 5, characterised in that the liposome is used to carry staurosporine.

12. The use according to claim 5, characterised in that the liposome is obtained by preparing a solution of a lipid mixture containing DPPC (1,2-dipalmitoyl-sn-glycero-3-phosphocholine) at a concentration of 17.0 mg/ml to 70.7 mg/ml, cholesterol at concentrations from 6.4 mg/ml to 18.6 mg/ml, DSPE-PEG(2000)amine (ammonium salt of 1,2-distearoyl-sn-glycero-3-phosphoethanolamine-N-[amino(polyethylene glycol)-2000]) from 3.7 mg/ml to 10.7 mg/ml and DOPC (dioleoylphosphatidylglycerol) from 0 mg/ml to 15.7 mg/ml and DOPG (dioleoylphosphatidylcholine) from 0 mg to 15.8 mg/ml in ethanol, heating the solution at 60° C. for 15 minutes, preparing an ammonium sulphate solution of 250 mM to 350 mM in water, fixing the pH between 2.62 and 5.5 by titrating with 0.2 M NaOH solution and finally mixing the lipid solution in ethanol with the ammonium sulphate solution in a volume ratio of 1:5 to 1:9.

13. The use according to claim 12, characterised by the addition of hydroxypropyl cyclodextrin (HPCD) at a concentration of 10 mg/ml to the ammonium sulphate solution.

14. The use according to claim 5, characterised in that the outer surface of the liposome is coated with a layer of human serum albumin (HSA).

15. A method of loading a liposome with drugs, especially with anticancer activity, characterised by the fact that into liposomes composed of at least three lipids, including DPPC (1,2-dipalmitoyl-sn-glycero-3-phosphocholine) in an amount of 48.6 to 70.7% by weight, cholesterol in an amount of 18.3 to 18.6% by weight, DSPE-PEG(2000)amine in an amount of 10.6 to 10.7% by weight, at least one drug is introduced in such a way that the liposomes are dialysed in buffer 0.1 M PBS with a pH of 7.4 to 9.0 from 12 h to 24 h to obtain a pH and concentration gradient, a solution of at least one drug in DMSO or water at a concentration of 50 mg/ml is added sequentially for every 1 ml of liposome solution, the solution is then cooled to 4° C. and allowed to stand from 4 h to 14 h or heated in a water bath for 15 to 60 minutes at 45 to 60° C., after which the resulting nanoparticles are dialysed in PBS buffer pH 7.4 at 4° C. or purified on a chromatography column and the drug concentration and the encapsulation rate of the drug into the liposomes are determined.

16. The method according to claim 15, characterised in that the liposome contains DOPC in an amount of not more than 1.77 mg/ml.

17. The method according to claim 15, characterised in that the liposome contains DOPG (dioleoylphosphatidylcholine) in an amount not exceeding 1.58 mg/ml.

18. The method according to claim 15, characterised in that the drug is emetine, introduced into solution as emetine hydrochloride at a concentration of 50 mg/ml in an amount of 5 to 80 ul per 1 ml of liposome solution.

19. The method according to claim 15, characterised in that the drug is mitoxantrone, introduced into solution as mitoxantrone dihydrochloride at a concentration of 50 mg/ml in an amount of 12.5 ul to 30 ul per 1 ml of liposome solution.

20. The method according to claim 15, characterised in that, after loading the drug into the liposomes, a 10% aqueous solution of human serum albumin (HSA) is added at a volume ratio of liposomes to HSA solution of 3:1.

21. The method according to claim 15, characterised by the fact that the dialysis of the nanoparticles is carried out in a chromatographic column filled with Sephadex G-25 or G-50.

22. The method according to claim 15, characterised in that the drug concentration and the encapsulation rate of the drug into the liposomes are determined by UV-Vis spectroscopy, HPLC with UV-Vis detection or by LC-MS.

23. The method according to claim 15, characterised in that the drugs are stabilised with hydroxypropyl cyclodextrin (HPCD).

24. A method of loading a liposome with drugs, particularly those having anticancer activity, characterised in that into liposomes composed of at least three lipids, including DPPC (1,2-dipalmitoyl-sn-glycero-3-phosphocholine) in an amount of 48.6 to 70.7% by weight, cholesterol in an amount of 18.3 to 18.6 wt %, DSPE-PEG(2000)amine in an amount of 10.6 to 10.7 wt %, at least one drug is introduced, the primary drug being staurosporine, such that the liposomes are dialysed in 0.1 M acetate buffer pH 4.5 to 5.5 for 2 h to 12 h at 4° C. to replace the external buffer, and then 20 to 50 ul of DMSO for each 1 ml of liposomes and 3.76 ul to 40 ul of staurosporine solution at 40 mg/ml in DMSO are added to the liposomes in divided doses, stirring each time until the precipitate disappears, after which the solution is left at room temperature for 15 to 60 minutes and the excess staurosporine is purified by dialysis in 0.1M PBS buffer, pH 7.4 to 8.5, changing the external buffer three times after 1 h, 6 h and 12 h.

25. The method according to claim 24, characterised in that the liposome contains DOPC in an amount of not more than 1.77 mg/ml.

26. The method according to claim 24, characterised in that the liposome contains DOPG (dioleoylphosphatidylcholine) in an amount not exceeding 1.58 mg/ml.

27. The method according to claim 24, characterised in that after loading staurosporine into liposomes, a 10% aqueous solution of human serum albumin (HSA) is added at a volume ratio of liposomes to HSA solution of 3:1.

28. The method according to claim 24, characterised in that the concentration of loaded staurosporine is determined by UV-Vis spectroscopy against a calibration curve by breaking up the liposome sample by the addition of 0.3M HCL in a volume ratio of 1:1 and successively 18 times the volume of ethanol.

29. The method according to claim 24, namely that to liposomes containing staurosporine after dialysis in PBS buffer, a solution of emetine hydrochloride of 50 mg/ml in water or DMSO is added in an amount of 10 ul to 33.4 ul per 1 ml of liposomes and then left for 6 h to 12 h at 4° C. or heated for 15 minutes at 45° C.-60° C., after which the % EE encapsulation rate of both drugs is determined taking advantage of the poor water solubility of staurosporine by separating the liposomes on the chromatography column by eluting the unbound drugs from the liposome successively with PBS buffer pH 7.4 and a solution of water:ethanol (1:1), and the remaining liposomes are purified by dialysis in buffer 0.1 M PBS or saline and the concentration is determined by UV-Vis spectroscopy against a calibration curve, or HPLC with UV detection, or by LC-MS.

30. The method according to claim 24, characterised in that the staurosporine is stabilised with hydroxypropyl cyclodextrin (HPCD).
